(19) United States
(12) Patent Application Publication
(10) Pub. No.: US 2004/0203158 A1
(43) Pub. Date: Oct. 14, 2004

(54) Transposon-insulator element delivery systems

(76) Inventors: Perry B. Hackett, Shoreview, MN (US); Scott McIvor, St. Louis Park, MN (US); Karl J. Clark, Ramsey, MN (US); Ljubica Caldovic, Silver Spring, MD (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 10/758,237

(22) Filed: Jan. 15, 2004

Related U.S. Application Data
(60) Provisional application No. 60/440,125, filed on Jan. 15, 2003.

(51) Int. Cl. C12N 15/74

(52) U.S. Cl. 435/473

(57) ABSTRACT

Certain embodiments are directed to using an insulator element in a transposon having at least one transcriptional unit and at least one insulator element. The transcriptional unit(s) may be flanked by at least one insulator element on each side. The transcriptional unit may include an exogenous nucleic acid for introduction into a cell, e.g., DNA encoding a marker molecule. The insulator element may include a binding site for a CTCF protein. And, for example, a transcriptional unit may be disposed between a first insulator element and a second insulator element, and the first insulator element and the second insulator element may be disposed between inverted repeats of a transposon. The exogenous nucleic acid may be, e.g., DNA encoding an antisense RNA or siRNA.

### CROSS-REFERENCE TO RELATED APPLICATIONS

This Application claims priority to U.S. provisional patent Serial No. 60/440,125, filed Jan. 15, 2003, which is hereby incorporated by reference herein.

### FIELD OF THE INVENTION

Some embodiments of the invention are in the field of the delivery of exogenous nucleic acids to cells, especially by use of transposons that incorporate insulator elements.

### BACKGROUND

Vectors used to deliver genes into chromosomes of humans often integrate in a near random fashion into chromatin. The problem of insertional mutagenesis occurring as a result of random integration of a genetic construct has been a long-term problem that has been recognized for many years (e.g., see Verma and Somia, 1997). Recently, the most serious concerns about this problem were fully realized when human patients died as a result of gene therapy treatments that triggered unwanted transcription of the patients' genes. These events apparently happened because transcriptional regulatory elements can act over long distances in chromatin, up to nearly 10,000 base pairs.

These events involved the use of retroviruses to correct deficiency of the common gamma chain in X-linked severe combined immunodeficiency (X-SCID). In these clinical trials, the retrovirus treatment resulted in a substantial therapeutic benefit to patients^{21,55} but unfortunately two of these patients have developed a leukemia-like syndrome. These adverse events are associated with integration of the retrovirus and activation of a nearby proto-oncogene, LMO2. As a result, in a precautionary measure, the Food and Drug Administration (FDA) placed on “clinical hold” all active gene therapy trials using retroviral vectors to insert genes into blood stem cells.

### SUMMARY OF PREFERRED EMBODIMENTS

The long-felt need to solve the problem of unwanted activation of host genes urgently requires a solution if gene therapy is to realize its clinical promise. Although the introduction of an exogenous gene into a host holds great promise for scientific study and medical therapies, improved methods for delivering nucleic acids are desired. In particular, a method that provides greater control of the effects on the native genome are desirable; in particular, approaches that do not activate, or stimulate expression of native genes. An approach to solve to this long-felt but unsolved need is set forth herein, and includes use of new gene vectors and use of factors in the vectors that stop unwanted transcription.

As described herein, insulating elements may be used to inhibit the unwanted transcription of host genes. Because neighboring genes in the host are individually regulated, there are borders that insulate the genes from one another. The borders are nucleic acid sequences referred to as insulating elements. This Application sets forth materials and methods for using the insulating elements in conjunction with transformation with transposons. Insulator elements appear to protect certain stretches of DNA from being transcriptionally silenced; for example, exogenous DNA has been introduced into a cell in conjunction with an insulator element to thereby prevent the transcriptional silencing of the exogenous DNA. It has not heretofore been appreciated, however, that exogenous insulating elements can be used to transcriptionally silence a gene of a host.

An embodiment for using an insulator element is a transposon having at least one transcriptional unit and at least one insulator element. The transcriptional unit(s) may be flanked by at least one insulator element on each side. The transcriptional unit may include an exogenous nucleic acid for introduction into a cell, e.g., DNA encoding a marker molecule. The insulator element may include a binding site for a CTCF protein. The insulator element may also include, e.g., at least one of SEQ ID NO:16, SEQ ID NO:17, OR SEQ ID NO:18, or structural and/or functional equivalents thereof. A transcriptional unit may be disposed between a first insulator element and a second insulator element, and the first insulator element and the second insulator element may be disposed between inverted repeats of a transposon. The exogenous nucleic acid may be, e.g., DNA encoding an antisense RNA or siRNA.

Other embodiments are directed to methods. For example, an embodiment is a method of altering a cell, the method involving exposing a cell to a transposon that has a transcriptional unit and at least one insulator element. The transcriptional unit may be flanked by at least one insulator element on each side of the transcriptional unit. A transcriptional unit may include an exogenous nucleic acid for introduction into a cell.

A transposon may be introduced into a cell as appropriate for the application, e.g., by electroporation or microinjection. Transposons with insulators may be introduced into any type of cell. Examples of such cells include lymphocytes, pancreatic cells, neural cells, muscle cells, blood cells, hepatocytes, hepatoma cells, primary hepatocytes, liver cells, stem cells, primary pancreatic cells, pancreatic stem cells, primary hematopoietic cells, and hematopoietic stem cells. Cells in vitro are contemplated. Cell sin living animals are also contemplated. Embodiments include introducing vectors into animals, including embryos, humans, humans, zebrafish, mice, and rats.

Another embodiment is a transposon having a transcriptional unit and a means for preventing regulation of transcription of host nucleic acid by the transcriptional unit following insertion of into a host mammalian cell nuclear genome. Such means are set forth herein, as will be understood by persons of ordinary skill in these arts after reading this application.

### BRIEF DESCRIPTION OF THE DRAWINGS

FIG. 1A depicts a host chromosome with two silent genes;

FIG. 1B depicts the mechanism of activation of host chromosome genes after insertion of a transcriptional unit that includes an exogenous gene with transcriptional enhancer sequences;

FIG. 2 depicts the blocking of insertional mutagenesis by the use of at least one insulator element;

FIG. 3 depicts an embodiment for inhibiting insertional mutagenesis following random insertion of transgenes by means of a suicide sequence nucleic acid, also referred to herein as a fail-safe suicide vector;

FIG. 4 depicts an embodiment wherein a nucleic acid sequence encoding a transposase 300 is introduced into a chromosome 100. The transposase is introduced with a suicide sequence nucleic acid 240, that can be triggered to destroy cells that incorporate the transposase and suicide sequence.

FIG. 5 depicts CTCF-enhancer-blocking motifs with respect to a transcriptional unit on a chromosome;

FIG. 6 represents the features of a test plasmid for use in testing of insulator elements using a transposon.

### DESCRIPTION OF PREFERRED EMBODIMENTS OF THE INVENTION ps Chromatin Structure, Insulator/Border Elements and Gene Expression in Vertebrates

The expression of subsets of genes in the various tissues of a vertebrate animal determines the function of that tissue. Although all intact genes in vertebrate genomes can be expressed, in any given cell of any given tissue, most genes are not expressed^{39,62}. Maintaining some genes in an active state while other genes are kept silent is essential for the proper functioning of the organism as whole. This is not a trivial problem in vertebrate cells because the regulators of gene expression often are spread out over tens of thousands of base pairs in the DNA³¹. Consequently, there is much to discover about how regulators of gene expression are constrained to act only on specific genes, which are expressed as transcriptional units. It is clear that the structure of the chromatin plays a critical role in maintaining the active or silent states of genes in eukaryotic organisms ranging from yeast to mammals. The structure of the chromatin depends on the presence, and interactions among, chromatin-binding proteins that regulate gene expression^{116,140}. The importance of chromatin structure for gene expression is also evident from genetic and transgenic studies¹³².

Transgenic animals were one of the initial products of recombinant DNA-based biotechnology^{56,73}. The main idea was to introduce genes into animal genomes in order to direct the production of specific proteins. In order to achieve this end, it was important that the introduced genes would be expressed at reliable levels through multiple generations of the transgenic animal. The principle concern was that expression the transgenes not be shut down. Thus, transgenic animals have been very important tools for studying regulation of expression of newly introduced genes.

Cis-acting regulatory elements, the sequences of DNA to which chromatin-binding proteins attach to affect transcription of associated genes, have traditionally been studied using transient assays in which cultured cells are transfected or embryos are microinjected with DNA that remains unintegrated into chromosomes. However, when constructs containing all of the regulatory elements identified in transient expression studies are integrated into chromosomes of animals, the expression of these transgenes often is different from what is expected¹⁵¹.

Many times the transgenes are either silent or expressed ectopically, that is, in tissues other than where expression was expected. Additionally, in animals that have multiple copies of a transgene in their genomes, overall expression levels of the transgenes are much lower than expected and not proportional to the their number in the chromosomes, which suggests that the genes are frequently turned off¹⁵¹. The dependence of expression of a transgene on the particular site of integration is presumably due to the influences of the neighboring chromatin^{5,14}. This site-dependent variability of expression of a transgene is called position effect. In contrast, transfers of genomic clones that contained genes flanked by extensive upstream and downstream regions many times resulted in position-independent expression of transgenes^{3,54,108,138}. The position-independent expression of transgenes flanked by DNA sequences that extended well beyond the identified transcriptional regulators indicated that in addition to the classical transcriptional regulators, known as enhancers and silencers, there are other types of DNA sequences that influence expression of genes integrated into chromosomes in an “all-or-none” manner. Transgenes are subject to equivalent position effects in all vertebrates, from zebrafish, a model system used by the inventor(s) to identify functions of genes and their regulatory units, to humans^{1,2,30,52,87}.

Organization of Genetic Material in the Cell Nucleus

While not being limited to a particular theory of operation, some general principles provide a useful conceptual framework. Genomic DNA in diploid mammalian cells is about two meters long and is packaged into a roughly spherical nucleus with an average diameter of 10 μm³². Therefore, chromatin has to be carefully compacted to fit into nuclei. The structure of chromatin is dynamic to support regulated expression of the tens of thousands of genes present in the DNA strands. Hence, interphase chromatin, the state of DNA with auxiliary proteins that exists when a cell is not replicating and dividing, should be organized so that transcriptional factors bound to enhancers and silencers interact only with the RNA polymerase complexes assembled at the promoters of the genes they are supposed to regulate. Cis-acting regulatory elements can be located up to tens of thousands of base pairs upstream and downstream from the coding region of the gene they regulate⁹⁵. When placed between two promoters enhancers can simultaneously activate transcription from both promoters^{18,128}. Therefore, mechanisms that prevent inappropriate promoter/enhancer interactions are necessary for the regulated expression of genes in eukaryotic cells.

Furthermore, chromosomes appear to be divided into domains of gene expression that are separated by insulator elements. One or more genes, with their transcriptional regulatory elements, can reside in a single domain. Insulator elements are DNA sequences that are believed to function by binding specific proteins to prevent regulation of gene expression in one domain by the transcriptional regulators of neighboring domains. Chromatin domains are probably either structural units helping to package DNA inside the nucleus, functional units that allow gene expression independent of the chromatin structure in the neighboring chromatin, or both^{50,127}. Thus, it is inferred that border elements are involved in regulated gene expression, as indicated by the results of genetic, biochemical and cytological examinations of chromatin structure and gene expression. Certain embodiments in this Application take advantage of these border element (also referred to herein as insulator elements) properties to make improved vectors for gene therapy and as research tools.

Genetic Evidence for Chromatin Domains

In addition to the evidence derived from cell organization data, there is genetic evidence that supports the importance of the chromosomal environment for gene expression. Studies in yeast, mammalian cells insect cells, and cells of other animals and plants show the existence of chromatin domains. For example, when the white gene, which normally resides in the euchromatin (portions of a chromosome in which genes can be expressed compared to heterochromatin that is comprised of DNA sequences essential for chromosome replication and segregation during cell division but which cannot support transcription of genes) translocates to a position near centromeric heterochromatin, its expression pattern is altered resulting in eyes with red or yellow patches¹²². This is called position effect variegation (PEV). PEV results from the spreading of heterochromatin into the translocated euchromatic gene in a portion of cells early in development³⁹. The heterochromatic or euchromatic state of the translocated gene is inherited in progenitor cells, leading to variegated eye-color phenotype. A similar phenomenon has been observed in the yeast, S. cerevisiae. Genes that were experimentally introduced near yeast telomeres are subject to repression by the telomeric heterochromatin¹¹⁶. Expression of ADE2 gene at its normal chromosomal position gives rise to white yeast colonies whereas inactivation of ADE2 gene results in red colonies. ADE2 gene, placed near telomeres, is expressed in some yeast cells while telomeric heterochromatin silences ADE2 gene in other cells⁵³. The silent or expressed state of ADE2 is inherited in progenitor cells giving rise to sectored, red and white, colonies. Repression of genes by telomeric heterochromatin is called “telomeric silencing”. Genes located up to four kilobases away from telomeres can be subject to telomeric silencing¹²¹. Sectored colonies in yeast and variegated eye-color in Drosophila suggest that the silent or expressed state of genes near heterochromatin, once established, can be maintained through many rounds of cell divisions. PEV and telomeric silencing support the hypothesis that the chromosomal environment plays an important role for maintenance of the expressed or the silent state of genes and that there are boundaries which prevent spreading of the heterochromatin into the euchromatic regions of genomes.

Genetic evidence for the existence of DNA sequences which prevent promoter/enhancer interactions comes from mutant phenotypes caused by insertions of gypsy retrotransposons between cis-acting regulatory elements and promoters of genes such as yellow, Ultrabithorax and cut^{67,98,111}. The mutant phenotypes can be reverted either by excision of the retrotransposon or by mutations in the su(Hw) gene. The portion of the gypsy transposon responsible for mutant phenotypes is an array of binding sites for Su(Hw) protein^{112,131}. The Su(Hw) protein also binds to sites in approximately 200 bands of polytene chromosomes that do not contain gypsy transposons^{60,110}. All of this indicates that there are DNA elements that bind specific proteins to insulate genes from activation or repression by neighboring transcriptional regulatory elements.

Cytological and Biochemical Evidence for Chromatin Domain—MARs

A significant body of research has been devoted to candidate matrix attachment regions (MARs). This research, when viewed in light of recent data about insulator elements, provides further insight into insulator element structure, function, and activity. A brief overview of this body of research, starting at the level of chromatin organization, is helpful for placing insights into insulator elements in context.

Chromatin is structured with several levels of organization. The first level of organization is the winding of DNA around core histones to yield nucleosomes⁷⁹. Nucleosomes coil into a solenoid structure to form what is called the 30 nm fiber that consists of six nucleosomes per turn of the coil. 30 nm fibers appear to have a ribbon-like, zigzag organization with linker DNA crisscrossing the axis of chromatin fiber^{153,154}. 30 nm fibers are coiled into higher order coils to give greater compaction of the DNA. The highest levels of organization can be seen in certain types of chromosomal structures called lampbrush chromosomes that were observed in early cytological studies of amphibian oocytes and polytene chromosomes in insects. Loops of chromatin of uniform thickness appear to be anchored to a protein complex that runs along the axis of chromosome¹²⁷. Transcribed genes, marked by nascent mRNA strands in electron micrographs, reside in the loops. In polytene chromosomes from fruit flies, which have reproducible pattern of bands that are thought to result from different degrees of chromatin compaction, transcriptional activation is accompanied by expansion of these bands into “puffs” that may reflect myriads of RNA tendrils emerging from the DNA templates. Such cytological studies suggested that chromosomes are subdivided into a series of domains, manifested as loops anchored to a network of proteins called either the nuclear matrix, the nuclear scaffold or the nucleoskeleton¹¹⁴. The nucleoskeleton extends throughout the nucleus⁶⁴. In one model derived from identifying sites of RNA polymerase II binding to chromatin, the activation of genes may determine chromatin organization¹²⁹. In an alternative model, special DNA sequences demarcate boundaries between loops. These sequences are presumably bound by nuclear matrix proteins that attach the loops to the matrix.

Several biochemical methods have been developed to isolate nuclear matrix proteins and identify the DNA sequences that bind nuclear matrix. Nuclei extracted with lithium diiodosalicilate to remove histones and the majority of other nuclear proteins produce DNA and what is called the nuclear scaffold⁹⁶. Chromosomal DNA sequences to which the proteins from nuclear scaffolds can bind are called scaffold attachment regions (SARs). Alternatively, another method of chromatin extraction using 2M NaCl yields what is called the nuclear matrix^{24,25}. Proteins associated with the nuclear matrix can be used to test other DNA sequences for binding in vitro to identify candidate matrix attachment regions (MARs). One might expect that SAR-binding proteins and MAR-binding proteins should be the same but few tests for identity of the two types of protein have been done, in part because both treatments are sufficiently harsh that there are some doubts about the integrity of the isolated matrix and scaffold proteins—both methods yield poorly defined mixtures of proteins that vary with modifications of the isolation protocols^{7,38}. Proteins identified as components of nuclear matrix or scaffold include topoisomerase II¹⁰, nuclear matrins^{6,58,104}, SATB1 from thymus cells^{29,34,103}, lamins A and B9^{0,91}, SAF-A from HeLa nuclei¹²⁵, attachment region-binding protein (ARBP)^{144,145} and p120/hnRNP¹⁴⁶. ARBP binds to specific sequence motif in 5′-MAR from the chicken lysozyme gene^{144,145}. The SAF-A and SATB1 proteins can bind cooperatively to many different MARs^{34,103,125}. MARs have been isolated from many organisms, ranging from yeast to humans^{17,26}. Since MARs reside between genes as well as near transcriptional regulatory elements, it has been hypothesized that different classes of MARs have different roles in gene expression⁸²: MARs located between genes are thought to act as border elements while MARs which reside near enhancers and silencers are proposed to participate in regulation of transcription^{25,27}.

The sequences of MARs proximal to different genes are not identical. The sequences of some MARs are AT-rich, implying specific conformational properties of the double helix such as curvature and different sizes of the major and minor grooves. Other MARs appear to share sequence motifs, such as ATTA and ATTTA, that are so short as to be highly distributed (e.g. every few hundred basepairs of DNA on average) even though they contain recognition sequences for topoisomerase II, one of the proteins associated with MARS. Thus, some matrix protein preparations may be contaminated with polypeptides that recognize general conformational features of DNA rather than any specific sequence motifs¹⁷. This may explain the lack of sequence similarity among MARs, the variations in protein composition of various MAR and SAR preparations, and thus clouds conclusions of various reported interactions between MARs, which we henceforth use to include SARs, and matrix proteins in vivo.

Gentler methods for isolating chromosomal structures have been achieved by encapsulating cells into agarose beads followed by lysis with physiological buffers^{68,72}. Cytoplasmic components and chromatin can be removed by electroelution to leave a structure called the nucleoskeleton. Major components of the nucleoskeleton are lamins⁶⁴. Functional RNA polymerases may be attached to the nucleoskeleton^{69,71,117}. In these preparations, multiple RNA polymerases appear to be clustered in foci where transcription takes place⁶⁵ suggesting that these nuclear structures reflect their natural states. Observations such as these lead to a model in which the function of the nucleoskeleton is similar to the function of the cytoskeleton in the cytoplasm^{28,70}. In addition, transcriptional factories are attached to the nucleoskeleton. Templates are somehow brought to the transcriptional factories and threaded along them during transcription. According to this model the chromatin loops are formed by virtue of template attachment to transcriptional factories. Therefore, chromatin loops are dynamic, rather than static structures¹²⁹. This model does not address the problem of inappropriate promoter/enhancer interactions.

Biochemical evidence supports the cytological evidence for division of chromatin into transcriptionally active and inactive domains. DNase and restriction endonuclease sensitivity studies used to probe modifications of nucleosomes in genes that are actively transcribed provide additional biochemical evidence for the existence of chromatin domains. Genes that are actively transcribed or poised for transcription reside in chromatin that is more susceptible to digestion with nucleases than chromatin containing genes that are not expressed¹⁴⁸. Regions of DNase-sensitivity encompass coding regions of genes and also extend into upstream and downstream flanking regions and may coincide with the location of MARs^{11,83,88}. The explanation for this is obvious, chromatin fibers that are being transcribed by RNA polymerases are expected to be less encumbered with chromosomal packaging proteins and therefore less condensed, allowing easy access of DNA binding proteins to transcriptional regulatory elements as well as nucleases. Nucleosomes in actively transcribed genes are usually hyperacetylated and have other modifications to their histones relative to genes that are not transcribed^{33,106,123,134}. Loss of transcriptional activity of a transgene is accompanied by DNA methylation and histone deacetylation and is prevented by insulators^{115,120}. These two biochemical lines of evidence support the cytological studies that show that the eukaryotic genomes are divided into functional domains. For the μ immunoglobulin gene, nuclear matrix attachment regions antagonize methylation-dependent repression of long-range enhancer-promoter interactions^{43,44}. The role of DNA sequences in organizing chromatin can be tested as well as applied in transgenic animals, as described next.

MAR-Type Insulator Elements

All of this information provides evidence that insulator elements play a significant role in MARs. Transgenic animals have been used to test the ability of MARs to function as insulator elements. MARs appear to be able to ensure proper expression of transgenes in terms of tissue-specificity and timing. The lysozyme gene is expressed in macrophages and in hen oviducts^{13,14}. In these two tissues, the chicken lysozyme gene resides in a region of open chromatin that extends 7 kb upstream and 4 kb downstream from the coding region of the gene⁸⁸. At the edges of the open chromatin region are MARs that are postulated to separate the chicken lysozyme domain from neighboring domains. The MAR located upstream of the lysozyme gene is called the A-element. Between the A-element and the lysozyme gene are enhancers and silencers that regulate lysozyme expression in the oviduct and in myeloid tissues¹³. The A-elements allow expected patterns of expression of transgenes in tissue culture and in mice, although position effects are sometimes observed^{13,14,15,133}. There is a significant body of research that describes the functional, structural, and detailed features of A-elements so that a person of ordinary skill in these arts is able to identify A-elements. In combination with the disclosures of this Application related to insulator elements, therefore, a person of ordinary skill in these arts can make, use, and identify A-type insulator elements.

Although the MARs from the chicken lysozyme gene enhance the expression of transgenes integrated into chromosomes, they have no effect in transient expression assays in which the genes are expressed from plasmids rather than chromosomes^{113,133}. When A-element and 3′-MAR sequences were removed from the transgenic constructs, the expression levels of the lysozyme gene remained proportional to the number of transgenes integrated into chromosomes but the transgenes were expressed ectopically¹⁶. Conversely, when parts of 5′-flanking region were removed from the transgenic constructs, A-element and 3′-MAR sequences prevented ectopic expression of the lysozyme transgene but the levels of expression were not copy number-dependent¹⁶.

The A/T-rich binding protein ARBP is an abundant nuclear protein with high affinity for MAR/SARs. ARBP is homologous to rat methyl-CpG-binding protein MeCP2 and may function in defining chromatin loops and domains bordered by MAR sequences¹⁴⁹.

Similarly, A-elements were placed upstream and downstream of the whey acidic protein (WAP) gene and introduced in mice. The temporal and spatial specificity of expression of the WAP transgene, flanked by A-elements, corresponded to that of the endogenous WAP gene, whereas transgene expression was disregulated in the absence of A-elements^{92,93}. However, as with the lysozyme gene, the expression levels of WAP transgenes flanked by A-elements were not proportional to the number of transgenes integrated into chromosomes. MARs flanking the mammalian alipoprotein B gene have been used to insulate mammalian reporter genes in mammalian cells⁷⁴ and when flanking the white gene of Drosophila were able to confer position-independent expression in flies, demonstrating that mammalian MARs were functional in insect cells¹⁰⁵. These results indicate that MARs can protect transgenes from the influences of neighboring enhancers and silencers but that additional regulatory elements need to be present for copy number-dependent transgene expression.

MAR-like sequences have been identified at the boundaries of the DNase-sensitive region of the human β-interferon (β-IFN) gene. When a HSP70.1 promoter-luciferase gene construct was flanked by β-IFN gene MAR sequences, the expression profiles were as expected but the levels were not copy number-dependent¹³⁹. These results support the idea that MARs can prevent interactions of promoters from transgenes with the enhancers and silencers from neighboring genes. Thus, either with their normal genes or with heterologous genes, MARs act as neutral boundaries that do not alter the expression pattern of genes they flank, but they do not seem to protect genes against repression when integrated into some regions of chromosomes^{92,97,139}. Thus, it is known that some MARs, and A-elements, contain insulator elements that satisfy the test criteria for an insulator element, as set forth herein. As such, a person of ordinary skill in these arts will immediately be able to recognize such insulator elements and, after reading this Application, will be able use them with embodiments of the inventions as set forth herein.

Border-Type Insulator Elements

Some researchers have reported on border-type insulator elements that appear to function by a different mechanism than do MARs. Some MARs, however, apparently contain insulator elements. Therefore the different mechanisms attributed to MARs as compared to insulator elements may be a result of DNA sequences in the MARs associated with structural functions rather than strictly biochemical functions as well as functions that govern DNA replication as well as transcriptional regulation, which is the responsibility of insulator motifs.

The scs/scs′-elements from the 87A7 heat shock locus, su(Hw) binding region from the gypsy retrotransposon and the DHS5 site from the chicken β-globin locus are DNA sequences capable of alleviating position effects in transgenic fruit flies^{23,76,126,141}. The 87A7 locus spans 14 kb and contains two hsp70genes, transcribed in opposite directions, that are flanked by special chromatin sequences (scs/scs′) elements. Scs and scs′ elements each have a nuclease-resistant core flanked by DNase-hypersensitivity (DHS) sites that correlate with the boundaries of transcriptional regions and therefore with the sites that are likely to harbor border element sequences^{12,35,65,83,84,148}. The position of DHS sites within the scs/scs′-elements changes upon heat shock induction of hsp70genes, suggesting that chromatin domains may be structure-based rather than sequence-based^{142,143}. Both DHS sequences harbor topoisomerase II-recognition sequences, are under torsional strain in vivo⁴⁴, and both bind proteins in a sequence-specific manner. The scs element binds the protein bangdoo⁵⁰. Scs′ element binds the protein complex called BEAF (border element-associated factor) that consists of alternatively spliced forms of BEAF32 protein^{61,157}. Antibodies raised against BEAF32 bind to about 100-150 bands on polytene chromosomes¹⁵⁷, suggesting the presence of insulators throughout the Drosophila genome. Another scs-binding protein, zeste-white-5 (Zw5), is able to block enhancer-promoter interaction by binding to a specific 24-bp sequence within the scs element⁴⁶. Thus, the scs/scs′ insulators appear to act in concert with specific DNA-binding proteins.

In transgenic fruit flies, scs/scs′-elements can insulate the white gene, driven by a minimal promoter, from activation by neighboring enhancers. This resulted in flies with pale yellow eyes⁷⁶. All transgenic flies had bright red eyes when scs/scs′-elements flanked a white gene equipped with all its regulatory elements, indicating that scs/scs′-elements protected the transgene from repression by silencers. The ability of scs/scs′-elements to prevent interactions between transcriptional regulatory elements and promoters was tested further in enhancer-blocking assays. Both elements prevented activation of transcription of transgenes when placed between a wide variety of enhancers and promoters⁷⁷ and in a multiplicity of chromosomal contexts, even in amphibian and zebrafish cells^{19,20,37,80}.

Protein-binding ability led to the identification of another insulating sequence, that in the gypsy transposable element of Drosophila. A DNA sequence in gypsy retrotransposons, which contains twelve binding sites for the Su(Hw) protein, was identified as an insulator because it can prevent the regulation of transcription of genes when the gypsy transposon lands between their promoter and cis-acting sequences^{67,98,111,126}. Other polypeptides like the mdg4 proteins may interact with su(Hw) to establish insulation^{45,49,51}. The su(Hw)-binding region protects transgenes from position effects and has the ability to block interactions between many different enhancers and promoters^{18,76,78,126}. Moreover, the gypsy insulator determines the nuclear localization of DNA; DNA sequences flanked by Su(Hw)-elements tended to be on the nuclear periphery introduced into Drosophila diploid imaginal disk cells⁴⁸. Thus, this activity correlates with cytological observations noted earlier. Surprisingly, insulation by Su(Hw)-binding sites is sensitive to the number of insulators. Two tandem copies of insulator Su(Hw) were ineffective in blocking various enhancers from activating a downstream promoter, a single enhancer was partially effective in blocking enhancer activation when placed between the promoter and enhancer, and flanking an enhancer with single insulators on each side was essentially completely effective in blocking its activity^{8,18a,99,101}. These data do not suggest that these insulating elements work by simple structural rearrangement of chromatin⁴⁷ nor by some sort of simple decoy mechanism⁵⁰. Rather, the data suggest that the border elements work in pairs and can cancel their boundary effects when placed close to one another, perhaps by a mechanism similar to that which occurs with transfection¹⁰⁰.

The DNA fragment which contains 5′ constitutive DHS from the chicken β-globin locus control region (LCR) also insulates the white gene from position effects in transgenic fruit flies⁸². The Felsenfeld laboratory^{8,9,22,23,41,102,119,150} and others^{85,86,147,155} have conducted detailed molecular studies of this locus. They have found that the gamma and beta-globin loci contain both properties associated with border elements and insulator elements. The first are barrier motifs that serve to block heterochromatization by spread of methylation that can permanently shutdown expression of transgenes. The second property is enhancer-blocking activity mediated by a CTCF-binding site such that enhancers neighboring a transgenic construct will not influence the activity of the transgene^{8,9,81,150}. CTCF-binding sites have been identified in other boundary elements^{42,75}.

Other examples of insulator elements include the sns insulator from the sea urchin arylsulfatase gene^{4,36}, sequences flanking the early H2A histone gene in sea urchins^{94,107}, the Fab-7 sequence in the Drosophila bithorax complex that insulates the iab-7 gene^{5,57,158}, the MAR sequences flanking the tyrosinase gene in mammals¹¹⁸, and the methylation-regulated⁶³ insulator sequences found in the H19/Igf2 locus^{59,75}.

In partial summary of information about insulator elements, as stated above, chromosomes appear to be divided into domains that contain either expressed or silent genes^{127,152}. These domains are presumably separated by DNA sequences that have a variety of names including insulator sequences and border elements¹²⁷. As used herein, however, the term “insulator element” encompasses nucleic acid sequences that have been referred to as border elements as well as insulator elements. Insulator elements appear to be able to prevent influences of neighboring chromatin on genes within a chromatin domain¹²⁷. Insulator elements can be identified as DNA sequences capable of alleviating position effects in transgenic animals. In this application, an insulator element refers to a DNA sequence that will block transcriptional regulatory sequences in a transgenic construct from acting on neighboring genes after the integration of the transgenic construct into a chromosome. Detailed test protocols for an insulator element are provided below. Certain embodiments are insulator elements have a size in the range of about 10 to about 2500 base pairs; a person of ordinary skill will realize that all ranges and values within the range specifically set forth are contemplated and included herein, e.g., about 20 to about 1250 base pairs, less than about 1000 base pairs, and about 20 to about 300 base pairs. Further, some insulator elements are known to comprise certain regions for binding factors to regulate transcription; accordingly, some embodiments are directed to those motifs, or essentially those motifs.

As set forth above, insulator elements have some common features. First, they are typically active only when part of chromatin. Second, insulator elements do not alter the expression levels of genes in transient assays. Third, neither MARs nor other insulator element types alter tissue-specificity of transgene expression. Essentially MARs appear to block enhancer activity, but do not have silencing-blocking activity. Many insulators have both. Because insulator elements have a role in establishing domains of open chromatin characterized by global changes in histone modifications, they have been employed in transgenic studies to protect transgenes on viral vectors from being silenced and/or from their expression being influenced by neighboring transcriptional regulatory sequences.

Insulator Elements in the Patent Literature

Some reports of insulators are set forth in the patent literature. For example, U.S. Pat. No. 6,395,549, entitled “Long terminal repeat, enhancer, and insulator sequences for use in recombinant vectors”, hereby incorporated by reference, describes use of an insulator element with a viral construct to control expression of exogenous genes introduced into a cell. And, for example, U.S. Pat. No. 5,731,178, entitled “Attachment-elements for stimulation of eukaryotic expression system” describes use of an insulator element, i.e., an attachment element, with a viral transfection construct. Similarly, other reports include U.S. Pat. No. 6,100,448, entitled “Increasing expression of transgenes in plant cells using insulator elements” and U.S. Pat. No. 5,610,053, entitled “DNA sequence which acts as a chromatin insulator element to protect expressed genes from cis-acting regulatory sequences in mammalian cells.”

No one heretofore, however, is believed to have proposed using insulator elements to protect resident chromosomal genes from activation by inserted exogenous and/or transgenic constructs. Indeed, these applications, above, are generally limited to the use of insulator elements to prevent repression of an exogenous gene in a host cell. In contrast, some embodiments set forth herein are directed to insulator elements for preventing transcription of native genes when an exogenous gene is introduced into a host cell. The unwanted transcription of native genes has been a long-felt need that has not been addressed. The use of insulator sequences as described herein, however, addresses this problem. One reason that insulator sequences have not been previously proposed as a solution to this problem is that insulator sequences are commonly understood as promoting the transcription of the introduced exogenous sequence. In contrast, as set forth herein, and without being limited to a particular theory of operation, it is understood that insulator sequences serve to create domains to which transcription factors are isolated. As a result, enhancers and/or promoters inserted into a host chromosome do not activate the transcription of nucleic acids outside of the domain. Thus, insulator elements can be used to isolate exogenous transcription sequences from native genes, and prevent the transcription of host nucleic acids.

Moreover, these patent applications, set forth above, are generally limited to the use of viral vectors. Certain embodiments herein, however, are directed to the use of transposon vectors. There are differences between transposonal vectors and viral vectors that would not lead a person of ordinary skill in these arts to substitute one for the other with respect to the use of an insulator element. For many applications involving genetic transfection, e.g., gene therapy, long-term expression of genes is essential. Transcriptional silencing of genes introduced by retroviruses poses a major obstacle to their use as gene therapy vectors, and border elements have been proposed as a solution¹⁰⁹. Consequently, a number of investigators have incorporated border elements into their constructs to block methylation of the therapeutic gene^{40,66,124,135}. Hackett has gone further to demonstrate, using zebrafish and viral transfections as a model system, that border elements can maintain reproducible levels of transgene expression in multiple generations.^{19,20}. However, none of these studies have addressed the blocking of enhancer effects of the transgenic constructs on endogenous chromosomal genes. Nor do these studies address introducing insulator elements with transposons. Transposons, in fact, are not subject to the same transcriptional silencing challenges that plague the viral vectors.

Insulator Elements for Use with Vectors

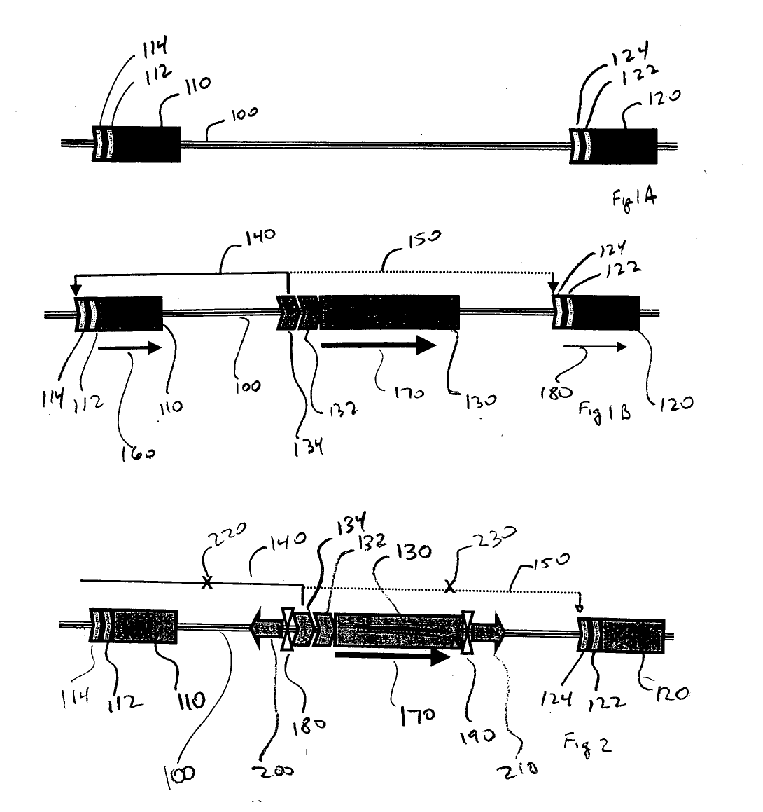

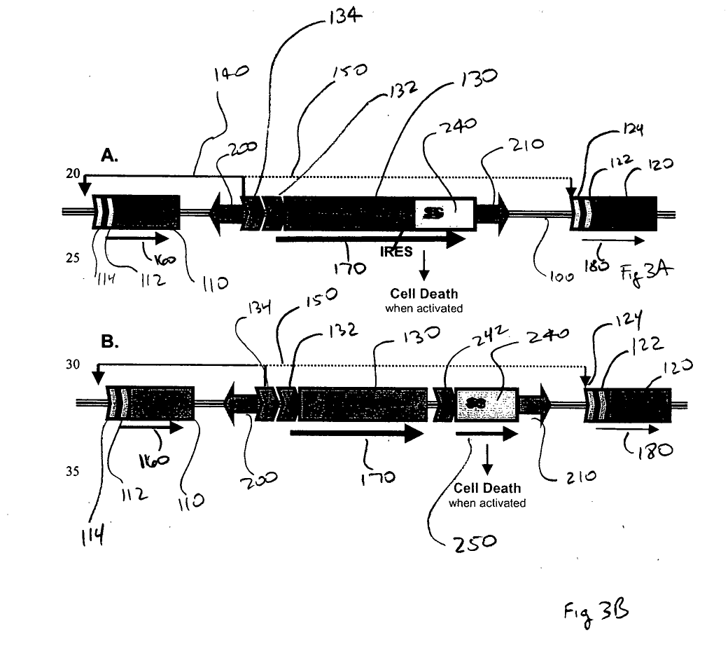

FIG. 1 depicts the phenomenon of insertional mutagenesis following random insertion of transgenes. The depiction FIG. 1 is simplified by neglecting the effects of three-dimensional structures and corresponding proteins and is a guide to the relevant issues at the level of DNA. Chromosome 100 having genes 110, 120 and the genes having promoters 112, 122 and enhancers 114, 124 is depicted in FIG. 1A, wherein genes 110, 120 are transcriptionally silent. Insertion of an exogenous therapeutic or marker sequence 130, along with promoter 132 and enhancer 134 may activate the nearby genes 110 or 120 via the enhancer 134, with the solid line 140 indicating a strong activation and the dotted line 150 indicating a weaker or nonexistent activation. Arrows 160, 170, 180 show expression of the genes, with the weight and length of the arrows indicating potential expression levels; with arrow 170 indicating the most expression, arrow 160 indicating lesser expression and arrow 180 indicating the least potential expression.

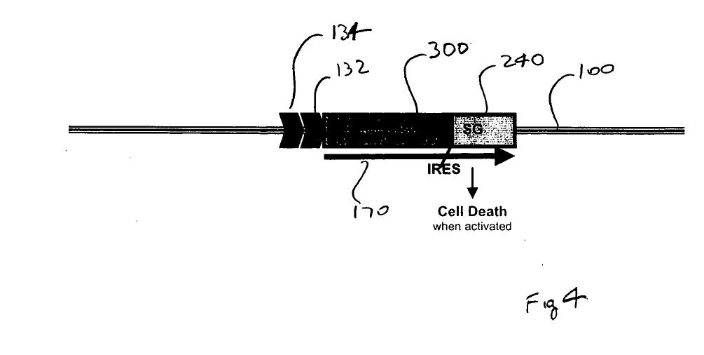

FIG. 2 depicts the blocking of insertional mutagenesis by the use of at least one insulator element. The inserted nucleic acid sequence 130 is flanked by insulator elements 180, 190, which are flanked by transposon inverted terminal repeat sequences 200, 210. The insulator elements block paths 140, 150, as indicated by Xs 220, 230. The transcription of exogenous nucleic acid sequence 130 is allowed but the expression of genes 110 and 120 is prevented. Alternatively, only one insulator element could be used, with that element preferably being placed as shown for element 180. The inverted terminal repeats of the transposon show how the vector can be designed for use with a transposon-based vector system.

FIG. 3 depicts an embodiment for inhibiting insertional mutagenesis following random insertion of transgenes by means of a suicide sequence nucleic acid, also referred to herein as a fail-safe suicide vector. As set forth in FIGS. 1 and 2, an exogenous gene 130 may be introduced into chromosome 100 using a transposon. A suicide sequence nucleic acid, e.g., a suicide gene, may also be introduced with gene 130. The suicide sequence 240 may be placed near gene 130, or further away, as shown in FIG. 3B. A promoter 242 may be incorporated into the chromosome 10 in a position to promote the suicide sequence 240, in which case expression of the suicide sequence would be increased, as indicated by arrow 250. The suicide sequence enables a triggering event to be used to kill cells transected with the sequence. For example, a substrate molecule may be supplied as a triggering event.

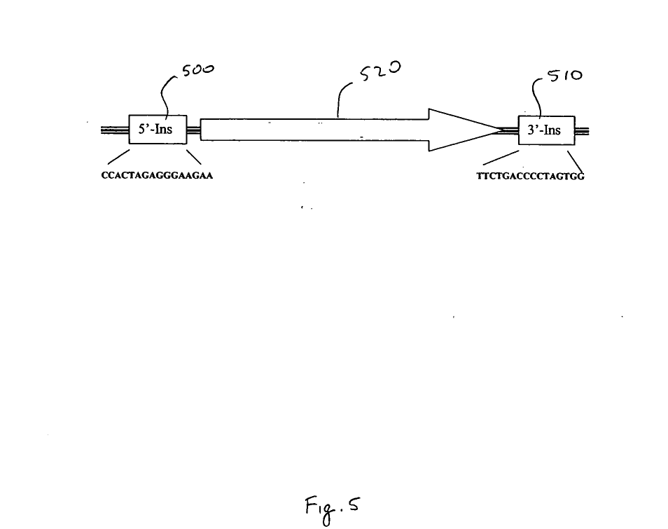

FIG. 4 depicts an embodiment wherein a nucleic acid sequence encoding a transposase 300 is introduced into a chromosome 100. The transposase is introduced with a suicide sequence nucleic acid 240, which can be triggered to destroy cells that incorporate the transposase and suicide sequence.

Vectors

Nucleic acids can be incorporated into vectors. As used herein, a vector is a replicon, e.g., a plasmid, phage, or cosmid, into which another nucleic acid segment may be inserted so as to bring about replication of the inserted segment. Vectors may be expression vectors containing an inserted nucleic acid segment that is operably linked to expression control sequences. An expression vector is a vector that includes one or more expression control sequences, and an expression control sequence is a DNA sequence that controls and regulates the transcription and/or translation of another DNA sequence. Expression control sequences include, for example, promoter sequences, transcriptional enhancer elements, and any other nucleic acid elements required for RNA polymerase binding, initiation, or termination of transcription. With respect to expression control sequences, the term operably linked means that the expression control sequence and the inserted nucleic acid sequence of interest (also referred to herein as the exogenous nucleic acid sequence that is intended to be expressed, also referred to as the exogenous nucleic acid sequence) are positioned such that the inserted sequence is transcribed (e.g., when the vector is introduced into a host cell). A transcriptional unit in a vector may thus comprise an expression control sequence operably linked to an exogenous nucleic acid sequence. For example, a DNA sequence is operably linked to an expression-control sequence, such as a promoter when the expression control sequence controls and regulates the transcription and translation of that DNA sequence. The term “operably linked” includes having an appropriate start signal (e.g., ATG) in front of the DNA sequence to be expressed and maintaining the correct reading frame to permit expression of the DNA sequence under the control of the expression control sequence to yield production of the desired protein product. Examples of vectors include: plasmids, adenovirus, Adeno-Associated Virus (AAV), Lentivirus (FIV), Retrovirus (MoMLV), and transposons.

There are a variety of promoters that could be used including, e.g., constitutive promoters, tissue-specific promoters, and inducible promoters. Promoters are regulatory signals that bind RNA polymerase in a cell to initiate transcription of a downstream (3′-direction) coding sequence.

Many different types of vectors are known. For example, plasmid vectors and viral vectors, e.g., retroviral vectors, are known. Mammalian plasmid expression vectors typically have an origin of replication, a suitable promoter and optional enhancer, and also any necessary ribosome binding sites, a polyadenylation site, splice donor and acceptor sites, transcriptional termination sequences, and 5′ flanking nontranscribed sequences. In addition, the expression vectors preferably contain a gene to provide a phenotypic trait for selection of transformed host cells such as neomycin resistance for eukaryotic cell culture, or tetracycline or ampicillin resistance in E. coli. Retroviral vectors, which typically transduce only dividing cells, can be used. Adenoviral vectors, capable of delivering DNA to quiescent cells can be used. Another viral vector system with potential advantages is an adeno-associated viral vector.

Transposonal Vectors

Nature uses two devices for introducing new genetic material into chromosomes of eukaryotic organisms. The first is viruses, against which most animals have defensive systems to protect their chromosomes from outside intruders. The second method used over long evolutionary periods (often millions of years) is to use transposons, which will enzymatically insert appropriate sequences of DNA into cellular chromosomes. However, merely introducing a gene into the genome of an animal is of little effect unless that gene is expressed to make a protein or confer some new property or function to the cell. So, it is useful to introduce transgenes into chromosomes in such a fashion that they can be expressed following delivery. Very often genes introduced by viruses are switched off soon after they enter chromosomes. This is a defense animals have against viruses. However, the Sleeping Beauty System is a non-viral method for delivery of individual genes into animal chromosomes. These newly introduced genes thus can be maintained and expressed over very long periods of time and can be passed from one cell generation to the next without loss of expression. Thus, by employing transposons and avoiding viruses, one has the ability to direct the integration of a wide variety of transgenes (foreign genes) into chromosomes of various vertebrate animals.

Transposons or transposable elements include a short piece of nucleic acid bounded by repeat sequences. Active transposons encode enzymes that facilitate the insertion of the nucleic acid into DNA sequences. In vertebrates, the discovery of DNA-transposons, mobile elements that move via a DNA intermediate, is relatively recent. Since then, inactive, highly mutated members of the Tc1/mariner as well as the hAT (hobo/Ac/Tam) superfamilies of eukaryotic transposons have been isolated from different fish species, Xenopus and human genomes. These transposable elements transpose through a cut-and-paste mechanism; the element-encoded transposase catalyzes the excision of the transposon from its original location and promotes its reintegration elsewhere in the genome. Autonomous members of a transposon family can express an active transposase, the trans-acting factor for transposition, and thus are capable of transposing on their own. Nonautonomous elements have mutated transposase genes but may retain cis-acting DNA sequences. These cis-acting DNA sequences are also referred to as inverted terminal repeats. Some inverted repeat sequences include one or more direct repeat sequences. These sequences usually are embedded in the terminal inverted repeats (IRs) of the elements, which are required for mobilization in the presence of a complementary transposase from another element.

A transposase is an enzyme that is capable of binding to DNA at sequences termed inverted terminal repeats. Transposons typically contain at least one, and preferably two, inverted repeats that flank an intervening nucleic acid sequence. The transposase binds to recognition sites in the inverted repeats and catalyzes the incorporation of the transposon into DNA. Transposons can be mobile, in that they can move from one position on DNA to a second position on the same or a different DNA molecule in the presence of a transposase. There are typically two components of a mobile cut-and-paste type transposon system, a source of an active transposase, and the DNA sequences that are recognized and mobilized by the transposase. Mobilization of the DNA sequences permits the intervening nucleic acid between the recognized DNA sequences to also be mobilized.

Cells that may be exposed to, or transfected by, transposons can be obtained from a variety of sources including bacteria, fungi, plants and animals, e.g., a vertebrate or an invertebrate; for example, crustaceans, mollusks, fish, birds, mammals, rodents, ungulates, sheep, swine and humans. Cells that may be exposed to a transposon include, e.g., lymphocytes, hepatocytes, neural cells, muscle cells, a variety of blood cells, and a variety of cells of an organism. Transposition is one of nature's methods for introducing new genetic material into chromosomes. Naturally, the process operates over evolutionary periods of time, i.e. hundreds of thousands to millions of years per transfer. As a result there are few, if any, defenses against transposition—the cost of defense is far too high for such infrequent benefit. Another natural method of introducing genes into chromosomes is via viruses; which in contrast to transposons pose constant threats to cells and therefore elicit host responses. As a consequence, transposons have been used for two general purposes in bacteria, yeast and lower animals such as insects and nematodes, gene transfer and gene discovery via insertional mutagenesis^{56,162-164}. The first application is as a non-viral transfer vector to direct the incorporation of specific nucleotide sequences into chromosomes. These sequences generally are fully able to express the genetic information carried in the transposon and thus represent a useful, non-viral method to achieve transfer of active genes. The second application of transposons is as an insertional mutagen¹⁶⁵⁻¹⁷⁰. In this case, a gene's function is interrupted by the integration of a transposon into the transcriptional unit. The power of insertional mutagens is dependent on the degree of the randomness of their integrations in genomic DNA. Transposons appear to integrate randomly¹⁶⁰.

For these applications, a source of transposase is required. Historically, transposase has been supplied via its gene that is transcribed to mRNA that then is translated into the transposase polypeptide. The transposase proteins then bind to the inverted terminal repeats for the cut-and-paste events that comprise transposition. In some instances, e.g., germ cells, mRNA encoding the transposase protein can be injected. For some systems injection of the transposase protein itself is done. By delivering transposase mRNA or protein, one can avoid the possible integration of the transposase gene, which in some circumstances is not desired. For nearly all cases, the source of transposons is a plasmid that contains the transposon. Plasmids are circular DNA molecules that are capable of being amplified to high numbers in appropriate bacteria such as Escherichia coli. For most purposes, up to 10¹² plasmids can be routinely harvested and purified from each ml of bacterial culture¹⁷³. Alternatively, viral vectors may be used to deliver a transposase, transposon, or both a transposase and a transposon.

The Sleeping Beauty Transposonal Vector

A particularly useful vector is a tranposase/transposon system for introducing nucleic acid sequences into the DNA of a cell, as set forth in U.S. Pat. No. 6,489,458 and U.S. patent Ser. Nos. 09/191,572 entitled “Nucleic Acid Transfer Vector For The Introduction Of Nucleic Acid Into The DNA Of A Cell”; Ser. No. 09/569,257 entitled “Vector-Mediated Delivery Of Integrating Transposon Sequences”; Ser. No. 10/128,998 entitled “Transposon System For Gene Delivery In Vertebrates”; and Ser. No. 10/128,998 “Composition For Delivery Of Compounds To Cells”; see also PCT application WO 99/25817, entitled “Nucleic Acid Transfer Vector For The Introduction Of Nucleic Acid Into The DNA of a Cell”, PCT Application WO 00/68399 “Vector-mediated delivery of integrating transposon sequences”; see also: Ivics, Z., P. B. Hackett, R. H. Plasterk and Z. Izsvak (1997). “Molecular reconstruction of Sleeping Beauty, a Tc1-like transposon from fish, and its transposition in human cells.” Cell 91: 501-510; Izsvak, Z., Z. Ivics and R. H. Plasterk (2000). “Sleeping Beauty, a wide host range transposon vector for genetic transformation in vertebrates.” J. Mol. Biol. 302: 93-10; Karsi A., B. Moav, P. B. Hackett and Z. Liu (2001) “Effects of insert size on transposition efficiency of the Sleeping Beauty transposon in mouse cells.” Mar. Biotechnol. 3: 241-245; Cui, Z., A. M. Geurts, G. Liu, C. D. Kaufmuan and P. B. Hackett (2002); “Structure-function analysis of the inverted terminal repeats of the sleeping beauty transposon.” J. Mol. Biol. 318: 1221-1235.; Harris, J. W., D. D. Strong, M. Amoui, D. J. Baylink and K. H. Lau (2002). “Construction of a Tc1-like transposon Sleeping Beauty-based gene transfer plasmid vector for generation of stable transgenic mammalian cell clones.” Anal. Biochem. 310: 15-26; Izsvak, Z., D. Khare, J. Behlke, U. Heinemann, R. H. Plasterk and Z. Ivics (2002). “Involvement of a bifunctional, paired-like DNA-binding domain and a transpositional enhancer in Sleeping Beauty transposition.” J. Biol. Chem.277: 34581-34588; Vigdal, T. J., C. D. Kaufman, Z. Izsvak, D. F. Voytas and Z. Ivics (2002). “Common physical properties of DNA affecting target site selection of Sleeping Beauty and other Tc1/mariner transposable elements.” J. Mol. Biol.323: 441-452; 8. Zayed, H., Z. Izsvak, D. Khare, U. Heinemann and Z. Ivics (2003). “The DNA bending protein HMGB1 is a cellular cofactor of Sleeping Beauty transposition.” Nucleic Acids Res. 31: 2313-2322; Geurts, A. M., Y. Yang, K. J. Clark, Z. Cui, A. J. Dupuy, D. A. Largaespada and P. B. Hackett (2003). “Gene transfer into genomes of human cells by the Sleeping Beauty transposon system.” Mol. Therap. 8: 108-117; Ivics Z., C. D. Kaufinan, H. Zayed, C. Miskey, O. Walisko, Z. Izsvak Z. (2004). “The Sleeping Beauty transposable element: evolution, regulation and genetic applications.” Curr. Issues Mol. Biol. 6: 43-55.; 11. Yant, S. R., and M. A. Kay (2003). “Nonhomologous-end-joining factors regulate DNA repair fidelity during Sleeping Beauty element transposition in mammalian cells.” Mol. Cell. Biol. 23: 8505-8518.; P. B. Hackett, and R. S. Mclvor. (2003). “Counterselection and co-delivery of transposon and transposase functions for the study of mediated transposition in cultured mammalian cells.” Som. Cell Mol. Genet. (in press); Izsvak, Z., S. E., Stuwe, D. Fiedler, A. Katzer, P. A. Jeggo and Z. Ivics (2004). Healing the wounds inflicted by Sleeping Beauty transposon by double-strand break repair in mammalian somatic cells. Mol. Cell. in press; Luo, G., Z. Ivics, Z. Izsvak and A. Bradley (1998). “Chromosomal transposition of a Tc1/mariner-like element in mouse embryonic stem cells.” Proc. Natl. Acad. Sci. USA 95: 10769-10773.; Dupuy, A. J., S. Fritz and D. A. Largaespada (2001). “Transposition and gene disruption in the male germline of the mouse.” Genesis 30: 82-88; Fischer, S. E., E. Wienholds and R. H. Plasterk (2001). “Regulated transposition of a fish transposon in the mouse germ line.” Proc. Natl. Acad. Sci. USA 98: 6759-6764.; Horie, K., A. Kuroiwa, M. Ikawa, M. Okabe, G. Kondoh, Y. Matsuda and J.Takeda (2001). “Efficient chromosomal transposition of a Tc1/mariner-like transposon Sleeping Beauty in mice.” Proc. Natl. Acad. Sci. USA 98: 9191-9196; Dupuy, A. J., K. Clark, C. M. Carlson, S. Fritz, A. E. Davidson, K. M. Markley, K. Finley, C. F. Fletcher, S. C. Ekker, P. B. Hackett, S. Horn and D. A. Largaespada (2002). “Mammalian germ-line transgenesis by transposition.” Proc. Natl. Acad. Sci. USA 99: 4495-4499.; Roberg-Perez, K., C. M. Carlson and D. A. Largaespada (2003). “MTID: a database of Sleeping beauty transposon insertions in mice.” Nucl. Acids Res.31: 78-81.; Carlson, C, A., Dupuy, S. Fritz, K. Roberg-Perez, C. F. Fletcher and D. A. Largaespada (2003). “Transposon mutagenesis of the mouse gerimline.” Genetics 165:243-256.; Horie, K., K. Yusa, K. Yae, j. Odajima, S. E. J. Fischer, V. W. Keng, T. Hayakawa, S. Mizuno, G. Kondoh, T. Ijiri, Y. Matsuda, R. H. A. Plasterk and J. Takeda (2003) “Characterization of Sleeping Beauty transposition and its application to genetic screening in mice.” Mol. Cell. Biol. 23: 9189-9207.; Yant, S. R., L. Meuse, W. Chiu, Z. Ivics, Z. Izsvak and M. A. Kay (2000). “Somatic integration and long-term transgene expression in normal and haemophilic mice using a DNA transposon system.” Nature Genetics 25: 35-41;Montini, E., P. K. Held, M. Noll, N. Morcinek, M. Al-Dhalimy, M. Finegold, S. R. Yant, M. A. Kay and M. Grompe (2002). “In Vivo Correction of MurineTyrosinemia Type I by DNA-Mediated Transposition.” Mol. Therapy 6: 759-769; Izsvak, Z., S. E., Stuwe, D. Fiedler, A. Katzer, P. A. Jeggo and Z. Ivics (2004). Healing the wounds inflicted by Sleeping Beauty transposon by double-strand break repair in mammalian somatic cells. Mol. Cell. in press; Luo, G., Z. Ivics, Z. Izsvak and A. Bradley (1998). “Chromosomal transposition of a Tc1/mariner-like element in mouse embryonic stem cells.” Proc. Natl.Acad. Sci. U S A 95: 10769-10773; Dupuy, A. J., S. Fritz and D. A. Largaespada (2001). “Transposition and gene disruption in the male germline of the mouse.” Genesis 30: 82-88; Fischer, S. E., E. Wienholds and R. H. Plasterk (2001). “Regulated transposition of a fish transposon in the mouse germ line.” Proc. Natl. Acad; Fischer, S. E., E. Wienholds and R. H. Plasterk (2001). “Regulated transposition of a fish transposon in the mouse germ line.” Proc. Natl. Acad. Sci. USA 98: 6759-6764; Horie, K., A. Kuroiwa, M. Ikawa, M. Okabe, G. Kondoh, Y. Matsuda and J. Takeda (2001). “Efficient chromosomal transposition of a Tc1/mariner-like transposon Sleeping Beauty in mice.” Proc. Natl. Acad. Sci. USA 98: 9191-9196; Dupuy, A. J., K. Clark, C. M. Carlson, S. Fritz, A. E. Davidson,. K. M. Markley, K. Finley, C. F. Fletcher, S. C. Ekker, P. B. Hackett, S. Horn and D. A. Largaespada (2002). “Mammalian germ-line transgenesis by transposition.” Proc. Natl. Acad. Sci. USA 99: 4495-4499; Roberg-Perez, K., C. M. Carlson and D. A. Largaespada (2003). “MTID: a database of Sleeping beauty transposon insertions in mice.” Nucl. Acids Res. 31: 78-81; Carlson, C, A., Dupuy, S. Fritz, K. Roberg-Perez, C. F. Fletcher and D. A. Largaespada (2003). “Transposon mutagenesis of the mouse germline.” Genetics 165:243-256; Horie, K., K. Yusa, K. Yae, j. Odajima, S. E. J. Fischer, V. W. Keng, T. Hayakawa, S. Mizuno, G. Kondoh, T. Ijiri, Y. Matsuda, R. H. A. Plasterk and J. Takeda (2003). “Characterization of Sleeping Beauty transposition and its application to genetic screening in mice.” Mol. Cell. Biol. 23: 9189-9207; Yant, S. R., L. Meuse, W. Chiu, Z. Ivics, Z. Izsvak and M. A. Kay (2000). “Somatic integration and long-term transgene expression in normal and haemophilic mice using a DNA transposon system.” Nature Genetics 25: 35-41. Montini, E., P. K. Held, M. Noll, N. Morcinek, M. Al-Dhalimy, M. Finegold, S. R. Yant, M. A. Kay and M. Grompe (2002). “In Vivo Correction of Murine Tyrosinemia Type I by DNA-Mediated Transposition.” Mol. Therapy 6: 759-769.; 3. Yant, S. R., A. Ehrhardt, J. G. Mikkelsen, L. Meuse, T. Pham and M. A. Kay (2002). “Transposition from a gutless adeno-transposon vector stabilizes transgene expression in vivo.” Nature Biotechnol. 20: 999-1005.; Ortiz, S., Q. Lin, S. R. Yant, D. Keene, M. A. Kay and P. A. Khavari (2003). “Sustainable correction of junctional epidermollysis bullosa via transposonmediated nonviral gene transfer.” Gene Therapy 10: 1099-1104.; Belur, L., J. L. Frandsen, A. Dupuy, D. H. Ingbar, D. L. Largaespada, P. B. Hackett and R. S. McIvor. “Integration and long-term expression in lung mediated by the Sleeping Beauty transposon system.” Mol. Therapy 8: 501-507.; Liu, G, Z. Cui, E. L. Aronovich, C. B. Whitley and P. B. Hackett (2003). “Excision of Sleeping Beauty transposons: parameters and applications to gene therapy.” J. Gene Medicine (in press); Mikkelsen, J. G., S. R. Yant, L. Meuse, Z. Huang, H. Xu and M. A. Kay (2003) Helper-independent Sleeping Beauty transposon-transposase vectors for efficient nonviral gene delivery and persistent gene expression in vivo. Mol. Therapy 8: 654-665.; Kren, B. T., S. S. Ghosh, C. L. Linehan, N. Roy-Chowdhury, P. B. Hackett, J. Roy-Chowdhury and C. J. Steer (2003). “Hepatocyte-targeted delivery of Sleeping Beauty mediates efficient gene transfer in vivo.” Gene Ther. Mol. Biol. 7: 229-38.; Izsvak, Z. and Z. Ivics (2004). Sleeping Beauty transposition: biology and applications for molecular therapy. Mol. Therap.: in press; Davidson, A. E., D. Balciunas, D. Mohn, J. Shaffer, S. Hermanson, S. Sivasubbu, M. P. Cliff, P. B. Hackett, and S. C. Ekker (2003). “Efficient gene delivery and gene expression in zebrafish using the Sleeping Beauty Transposon.” Dev. Biol. 263:191-202.; Grabher C, T. Henrich, T. Sasado, A. Arenz, J. Wittbrodt and M. Furutani-Seiki (2003). “Transposon-mediated enhancer trapping in medaka.” Gene. 322: 57-66.; Clark, K. J. A. M. Geurts, J. Bell, and P. B. Hackett (2004). “Transposon vectors for gene-trap insertional mutagenesis in vertebrates” (submitted); Hackett, P. B., S. C. Ekker and J. J. Essner (2003). Applications of transposable elements in fish for transgenesis and functional genomics. In Fish Developmental Biology and Genetics. (Zhiyuan Gong and Vladimir Korzh,eds.) (in press); Li, Z. H., D. P. Liu, J. Wang, Z. C. Guo, W. X. Yin and C. C. Liang (1998). “Inversion and transposition of Tc1 transposon of C. elegans in mammalian cells.” Somat. Cell. Mol. Genet. 24: 363-369.; Plasterk, R. H. A., Z. Izsvak and Z. Ivics (1999). “Resident aliens: the Tc1/mariner superfamily of transposable elements.” Trends Genet. 15: 326-332.; Richardson, P., C. Thoma, B. T. Kren and C. J. Steer (2002). “Strategies for hepatic gene correction.” J Drug Target 10: 133-141; Richardson, P. D., L. B. Augustin, B. T. Kren and C. J. Steer (2002). “Gene repair and transposon-mediated gene therapy.” Stem Cells 20: 105-118.; Kren, B. T., N. R. Chowdhury, J. R. Chowdhury and C. J. Steer (2002). “Gene therapy as an alternative to liver transplantation.” Liver Transpl. 8:1089-1108; Miskey, C., Z. Izsvak, R. H. A. Plasterk and Z. Ivics (2003). “The Frog Prince: a reconstructed transposon from Rana pipiens with high transpositional activity in vertebrate cells.” Nucleic Acids Res. 31: 6873-6881; each of which is hereby incorporated by reference herein, including all nucleic acid sequences and references thereto.

The Sleeping Beauty system is the first transposon system designed to integrate foreign DNA (called transgenic DNA) efficiently into vertebrate chromosomes. The Sleeping Beauty system involves the cooperation of the Sleeping Beauty transposases with inverted terminal repeats on the transposon. The repeats are capable of specifically binding to the transposase and are required for the transfection process to occur. Specifically bind is a term that refers to a binding event that involves a stereotyped interaction between two components and is a term of art that distinguishes such events from non-specific, or background binding. For example, CTCF specific binding to various elements has been documented, e.g., reference 41.

In an embodiment of the gene transfer system herein, the Sleeping Beauty (SB) protein can be introduced into the cell as a protein or as nucleic acid encoding the protein. In one embodiment the nucleic acid encoding the protein is RNA and in another, the nucleic acid is DNA. Further, nucleic acid encoding the Sleeping Beauty protein can be incorporated into a cell through a viral vector, anionic or cationic lipid, or other standard transfection mechanisms including electroporation, particle bombardment or microinjection used for eukaryotic cells. Following introduction of nucleic acid encoding Sleeping Beauty, the nucleic acid fragment of this invention can be introduced into the same cell.

Similarly, the nucleic acid fragment can be introduced into the cell as a linear fragment or as a circularized fragment, preferably as a plasmid or as recombinant viral DNA. Preferably the nucleic acid sequence comprises at least a portion of an open reading frame to produce an amino-acid containing product. In a preferred embodiment, the nucleic acid sequence encodes at least one protein and includes at least one promoter selected to direct expression of the open reading frame or coding region of the nucleic acid sequence. The protein encoded by the nucleic acid sequence can be any of a variety of recombinant proteins new or known in the art. In one embodiment the protein encoded by the nucleic acid sequence is a marker protein such as GFP, chloramphenicol acetyltransferase (CAT), beta-galactosidase (lacZ), and luciferase (LUC). In another embodiment, the protein encoded by the nucleic acid is a growth hormone, for example to promote growth in a transgenic animal, or insulin-like growth factors (IGFs).

Transposonal and Viral Vectors

There are some distinct differences between the use of viruses and transposons for gene transfection. One difference is that a transposon works in conjunction with a transposase. A transposase is a protein that controls removal and insertion of the transposon DNA. The presence of a transposon, by itself, will not cause transfection of a gene unless the transposase is present in the same cell as the transposon. Further, the transposase must be capable of binding to the transposon and cooperating with it to accomplish transfection.

Some aspects of successful gene therapy are to 1) find the appropriate gene for transfer, 2) find a method for delivery of the gene to the tissues that are affected or to other tissues that can provide the necessary activity from afar, and 3) achieve long-term expression of the transgene so that repeated deliveries are not required. 4) Above all, the gene must be introduced and expressed in a reliably safe manner. Viruses invade cells and in some cases deliver their genetic material to chromosomes. But, because there are so many viruses to which we are exposed, animals have developed elaborate defensive mechanisms to protect against most viral infections. As a result, genes delivered by viruses often are silenced soon after delivery, necessitating multiple therapeutic infections. But, multiple deliveries of viruses commonly elicit immunological responses that can be harmful and even lethal to the patient.

One conventional approach to addressing the silencing of virally delivered genes is to use insulator elements. Indeed, the elements have been used to enhance the expression of the virally delivered genes in some circumstances. A transposon, however, avoids the silencing of virally-delivered genes, and can be maintained and expressed over very long periods of time, and can be passed from one cell generation to the next without loss of expression. A transposon, therefore, would not conventionally be expected to be useful in combination with an insulator. As described herein, however, an insulator may advantageously be used to silence host genes, even with a transposon. Indeed, one advantage of the transposon system is that it avoids the use of viruses. Genes delivered into the livers of mice using, for example, the Sleeping Beauty Transposon System can partially restore deficiencies of several enzymes (Yant et al., 2000; PCT WO 01/30965).

Administration

One aspect of employing a transposon system in gene therapy is devising a mechanism for its delivery. One common method is the hydrodynamic delivery wherein a relatively large volume of transgenic DNA is injected into the circulatory system (the tail vein in mice) under high pressure—most of this DNA winds up in cells of the livery . Another method is to use negatively charged liposomes containing galactocerebroside, or complexed with lactosylated polyethyleneimine (PEI), which have been effective in delivering nucleic acids into hepatoma cells, primary hepatocytes and liver cells in living mice¹⁷².

The delivery of transposons to any tissue in the body is contemplated, including cells found in blood, liver, lung, pancreas, muscle, eye, brain, nervous system, organs, dermis, epidermis, cardiac, and vasculature. Delivery may be by, e.g., direct injection into or near the desired tissue, complexation with molecules that preferentially or specifically bind to a target in the desired tissue, control release, oral, intramuscular, and other delivery systems that are known to those skilled in these arts. Another embodiment for delivery is electroporation, e.g., electroporation of cells in the blood using an electroporator. Cells may be microinjected or electroporated in vitro or in vivo; detailed materials and methods for such processes are provided in U.S. patent Ser. No. 60/513,052, filed Oct. 21, 2003, entitled “Materials and Methods of Using Transposons Encoding RNAi”, which is incorporated by reference herein in its entirety. RNAi is described in greater detail elsewhere, e.g., see below, and in Yin and Wan (J. Q. Yin a nd Y. Wan. International Journal Of Molecular Medicine, (2002), 10: 355-365). As categorized by Yin and Wan, RNAi includes long double stranded RNAs, long single stranded sense RNA, single stranded RNAs that form duplexes, short double stranded RNAs, and short antisense RNAs. RNAi is the subject of U.S. Patent and PCT applications, e.g., certain of the following: US20030125281; US20030130186; US20030124513; US20030119017; US20030144239; US20030166282;, US20030148519; US20030157691; US20030153519; US20030139363; US20030166512; US20030036056; WO03056022; WO03020931; WO03008573; WO0244321; WO03070895; WO03070193; WO03070750; WO03070918; WO03070914; WO03066650; WO03068797; WO02097114; WO9946372; WO0060115; WO9519788; WO9206988; and U.S. Pat. No. 6,562,570 U.S. Pat. No. 5,985,661. U.S. Pat. No. 5,750,380 U.S. Pat. No. 5,750,380 U.S. Pat. No. 5,272,262 U.S. Pat. No. 5,149,796; U.S. Pat. No. 5,144,019; and U.S. Pat. No. 5,110,802. Use of RNAi and other materials and methods as described in these publications is contemplated in combinations with the embodiments described elsewhere herein.

Examples of delivery of certain embodiments herein include via injection, including intravenously, intramuscularly, or subcutaneously, and in a pharmaceutically acceptable carriers, e.g., in solution and sterile vehicles, such as physiological buffers (e.g., saline solution or glucose serum). The embodiments may also be administered orally or rectally, when they are combined with pharmaceutically acceptable solid or liquid excipients. Embodiments can also be administered externally, for example, in the form of an aerosol with a suitable vehicle suitable for this mode of administration, for example, nasally. Further, delivery through a catheter or other surgical tubing is possible. Alternative routes include tablets, capsules, and the like, nebulizers for liquid formulations, and inhalers for lyophilized or aerosolized agents.

Presently known methods for delivering molecules in vivo and in vitro, especially small molecules, nucleic acids or polypeptides, may be used for the embodiments. Such methods include microspheres, liposomes, other microparticle vehicles or controlled release formulations placed in certain tissues, including blood. Examples of controlled release carriers include semipermeable polymer matrices in the form of shaped articles, e.g., suppositories, or microcapsules and U.S. Pat. Nos. 5,626,877; 5,891,108; 5,972,027; 6,041,252; 6,071,305, 6,074,673; 6,083,996; 6,086,582; 6,086,912; 6,110,498; 6,126,919; 6,132,765; 6,136,295; 6,142,939; 6,235,312; 6,235,313; 6,245,349; 6,251,079; 6,283,947; 6,283,949; 6,287,792; 6,296,621; 6,309,370; 6,309,375; 6,309,380; 6,309,410; 6,317,629; 6,346,272; 6,350,780; 6,379,382; 6,387,124; 6,387,397 and 6,296,832. Moreover, formulations for administration can include transdermal patches, ointments, lotions, creams, gels, drops, suppositories, sprays, liquids, and powders.

Activation-safe Transposons

In another embodiment, an Activation-safe transposon includes a Sleeping Beauty Transposon System having a gene that can direct the demise of a cell in which either the therapeutic gene or the transposase gene is expressed (see FIGS. 3 and 4). Such a gene is colloquially referred to as a “suicide gene (SG).” The SG is expressed whenever the transgene is expressed. The term suicide sequence nucleic acid is used herein to refer to nucleic acid fragments that encode triggerable elements for cellular destruction. Embodiments herein that refer to a suicide gene will therefore be understood as also being applicable to a suicide sequence nucleic acid. Should an adverse event occur in a patient that is due to insertion of the therapeutic sleeping beauty vector, a substrate for the SG can be administered to the patient that will cause the product of the SG gene to kill the cells in which it is expressed. This procedure will undo the benefits of the therapeutic gene and can be used whenever the problems of the therapeutic construct are greater than the benefits.

For example, the Thymidine Kinase (TK) gene of the Human Herpes Simplex Virus is placed behind a marker gene (e.g., neo or Green Fluorescent Protein) or a therapeutic gene (e.g., glucuronidase B, GUSB) through an Internal Ribosome Entry Site (IRES) derived from the Encephalomyocarditis Virus (EMCV). As a result, two genes are co-transcribed as a single mRNA from the enhancer-promoter cassette driving the therapeutic gene. The EMCV IRES sequence allows expression of the TK gene, generally at a lower rate. The EMCV IRES element is an often-used sequence for this purpose (e.g., Fahrenkrug et al., 1999). The HSV TK protein will not affect cells unless the drug gangcyclovir is added, at which time the HSV TK gene converts the drug into a poisonous material that kills the cell in which the HSV TK gene is expressed. The HSV TK gene has been used for this purpose (Borelli et al., 1988) and has been proposed for gene therapy as a killing agent for cancer cells (e.g., Anderson, 1998). The suicide gene can be co-expressed with the transgene via a common promoter, with the IRES allowing translation of the suicide gene. Alternatively, the suicide gene can be independently transcribed from a separate promoter that can be independently activated. These two examples are shown in FIG. 3.

Other suicide sequence nucleic acids have been described in the patpetner literature, as in, for example: EP 0 694 070 Recombinant alphavirus vectors; WO 95/07994 Recombinant alphavirus vectors; EP 0 777 739 Genetic therapy of vascular diseases with a cell-specific active substance which is dependent on the cell cycle; EP 0 804 601 Genetic therapy of tumors with an endothelium cell-specific active substance which is dependent on the cell cycle; and EP 0 807 183 Genetic therapy of diseases caused by the immune system, said therapy using a cell-specific active substance which is dependent on the cell cycle.

The SG can also be attached to the Sleeping Beauty transposase gene to preclude its activity after initial transposition has occurred. This process relieves concerns that a transposase gene integrated into chromosomes of host cells could give a low level transposase activity that might lead to transformation of the cell in deleterious ways. Suicide genes include, but are not limited to, HSV TK, yeast or bacterial cytosine deaminase, microbial purine nucleoside phosphorylase, or any other gene encoding an enzyme that activates an inert substance to a product that is toxic in a cell in which it is expressed.

Embodiments include a transposon containing a genetic sequence (e.g., insulator sequence). Such elements/sequences may inhibit or block the activities of genetic signals outside the transposon from acting on genetic sequences inside the transposon. Other embodiments include a transposon containing genetic sequences that can direct the synthesis of a product that can kill a cell when activated by a substrate molecule. Other embodiments include a suicide gene attached to SB transposase gene. Other embodiments include a combination of a border element, insulator sequence, a suicide gene and their use on a transposon, a transposase, or both. Variations of the embodiments include use of the transposon with and without transposase (either as a gene, mRNA or protein), and use of transposase in trans (separate plasmid as transposon) and cis (on the same plasmid) configurations.

As an example, insulator elements may include, but are not limited to natural elements (e.g., HS4, chicken lysozyme A elements, Drosophila scs/scs′) and synthetic derivatives thereof. A natural element is an element found in nature but a synthetic element is not found in nature. Both natural and synthetic elements may be synthesized using, e.g., a machine. Border elements may include, but are not limited to natural elements (e.g., HS4, chicken lysozyme A elements, Drosophila scs/scs′) and synthetic derivatives thereof.

Activation-safe transgenic vectors, e.g., transposons and/or viruses, overcome problems wherein randomly inserted genetic material may deleteriously affect expression of genes residing in chromosomes. Viral vectors or non-viral vectors such as Sleeping Beauty transposons are used. Set forth herein are some examples of applications to overcome potential adverse effects of transgenic DNA (Milot et al., 1996). A notable application is in human gene therapy, where adverse events due to unintended activation of genes that caused problems to the patients. Activation-safe gene therapy vectors that contain insulator elements generally do not induce inappropriate expression of chromosomal genes outside the vector itself. The fail-safe version of gene therapy vectors, e.g., FIG. 3, allow clinicians treating a patient “to pull the plug” in a patient in which the gene therapy vector causes unintended adverse effects.

Additional Examples, Methods, and Uses

Certain embodiments described below involve the Sleeping Beauty transposon system that will either keep enhancers within the transposon from activating genetic sequences outside of the transposon or allow investigators/physicians to kill those cells that harbor a transposon that has activated a nearby gene(s). Some embodiments allow transposase activity to be controlled and are sometimes referred to herein as Activation-Safe Transposons and Fail-Safe Transposons.

Some embodiments include a transposon that includes an insulator sequence. Such a transposon may further include an exogenous DNA sequence. An exogenous sequence is a sequence that is intended to be introduced into an animal, a cell, a nucleus, or into another DNA sequence. An exogenous sequence may be natural or synthetic and may be naturally present in the animal that receives the exogenous sequence or it may be non-native to the animal. Examples of an exogenous sequence include a promoter, an enhancer, a marker sequence, a sequence encoding a therapeutic protein or a catalytic RNA, and insulator/border sequences.

Further examples include labeled nucleic acid sequences and nucleic acid fragments that encode markers. Labeled nucleic acid sequences are detectable, e.g., by fluorescence, nuclear magnetic resonance imaging, X-ray, microscopy, transmission electron microscopy, light microscopy, histological examination, and ELISA. A marker is a factor that allows for expression of a nucleic acid sequence to be detected, e.g., a green fluorescent protein, florescent molecules, enzymes, biotin, avidin, antibodies, and skin cell pigments.

Other examples of applications include the delivery of marker nucleic acid sequences to cells, including cells in plants and animals (including humans) as well as cells that are in cell culture, including organ or tissue culture, and including prokaryotic and eukaryotic cells. For example, a marker sequence of green fluorescent protein associated with at least one border elements/insulator sequence may be delivered to a eukaryotic cell in a petri dish.

A transposon having an exogenous sequence may also contain an enhancer and a promoter. Embodiments also include a transposon having an exogenous gene that encodes a suicide sequence nucleic acid, e.g., a suicide gene, or other sequence that causes the demise of a cell or a cluster of cells, as described below. The suicide sequence may also be used in combination with the insulator element. Embodiments set forth herein can be used in combination with both viral (e.g. lentiviruses, see, e.g., U.S. Pat. No. 6,013,516) and non-viral vectors (e.g., transposons, see, e.g., U.S. Pat. No. 6,489,458).

One embodiment of the activation-safe insulator transposon includes a Sleeping Beauty Transposon System with at least one, e.g., two, insulator elements, see FIG. 2. The insulator elements preferably are just inside the Inverted Terminal Repeats of an Sleeping Beauty transposon. The insulator elements act to block activation of genes proximal to the insertion site of the gene therapy transposon. Any of several types of insulator elements can be used. The structure and characteristics of insulator elements are set forth in greater detail in, for example, U.S. Pat. Nos. 5,731,178; 6,100,448; 6,395,549; 5,610,053, and PCT WO 00/23606, and references Chung et al., 1997; Recillas-Targa et al., 1999; and Bell et al., 1999. The inclusion of insulator elements does not affect the choice of therapeutic gene. The insulator elements may have the additional value of limiting “position effects” by neighboring, resident enhancers. Endogenous transcriptional enhancers can affect expression of an inserted gene in the same way that the inserted enhancers of a therapeutic gene can affect expression of resident genes. In this way the output of the therapeutic gene is more reliable.

Another example involves transgenic animals used as bioreactors for the manufacture of biological reagents, proteins, of medical value (Jaenisch, 1988; Dove, 2000). Transgene expressed from vectors similar to those used for human gene therapy direct the synthesis of the commercially valuable proteins. Just as the health of a human is important, so for animal bioreactors the health of the animal (cow, sheep, goat, pig, fish) is important. The activation-safe vectors can be used for transgenesis of animals that will result in reliable transgene expression without collateral damage to the animal because the embodiments designed for humans generally work in all vertebrate animals (Hackett and Alvarez, 2000).

Another example involves the use of transgenic fish that have been developed as a commercial food (Niiler, 2000). There are concerns of the effects of these fish, not only in terms of their augmented traits, but also in terms of unintended consequences of genetic engineering (Reichardt, 2000; Muir and Howard, 2002). Fish appear to be the first of several animals that will be genetically engineered for one or more particular traits. Activation-safe vectors can be used for transgenesis of animals that will result in reliable transgene expression without collateral damage to the animal because the embodiments designed for humans work in all vertebrate animals.

Another example relates to transgenic animals, which are extensively used for research applications. Functional genomics, the area of genomic science that seeks to attribute function to newly found genes from genome projects, depends on inactivation of genes as well as overexpression of genes. Overexpression of genes involves transgenesis (e.g., Camper, 1987). Activation-safe vectors can be used for transgenesis of animals that can result in reliable transgene expression with fewer or no effects on other endogenous genes that would obscure the functional significance of the transgene in a model vertebrate animal.

Another example relates to transgenic animals THAT have been deployed as sentinels for environmental toxins (e.g., Amanuma et al. 2000; Carvan et al., 2000; Winn et al., 2000). Transforming a natural animal into a bio-sentinel generally involves transgenesis. Activation-safe vectors can be used for making bio-sentinels without effects on other endogenous genes that are important for natural physiological pathways in the animal that could affect the performance and reliability of the bio-sentinel.

Another example relates to transgenesis in tissue culture to investigate the effects of transgenes. Implicit in the analyses is the assumption that the observed effects are due to the transgene and its intended activity (e.g., Hackett et al. 1999). Activation-safe transgenic vectors will give greater confidence in this fundamental assumption. Markers, therapeutic sequences, and sequences for producing a product may be used. Constructs for human gene therapy are first conducted in cultured cells and then in animals (e.g., Yant et al. 2000, 2002; Montini et al. 2002).

Other examples of applications include the delivery of marker nucleic acid sequences to cells, including cells in plants and animals (including humans) as well as cells that are in cell culture, including organ or tissue culture, and including prokaryotic and eukaryotic cells. For example, a marker sequence of green fluorescent protein associated with at least one border elements/insulator sequence may be delivered to a eukaryotic cell in a petri dish.

Nucleic Acids

As used herein, the term nucleic acid refers to both RNA and DNA, including cDNA, genomic DNA, synthetic (e.g., chemically synthesized) DNA, as well as naturally occurring and chemically modified nucleic acids, e.g., synthetic bases or alternative backbones. A nucleic acid molecule can be double-stranded or single-stranded (i.e., a sense or an antisense single strand). An isolated nucleic acid refers to a nucleic acid that is separated from other nucleic acid bases that are present in a genome, including nucleic acids that normally flank one or both sides of a nucleic acid sequence in a vertebrate genome (e.g., nucleic acids that flank a gene). The term isolated as used herein with respect to nucleic acids also includes non-naturally-occurring nucleic acid sequences, since such non-naturally-occurring sequences are not found in nature and do not have immediately contiguous sequences in a naturally occurring genome.

An isolated nucleic acid can be, for example, a DNA molecule, provided at least one of the nucleic acid sequences normally found flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences as well as DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not considered an isolated nucleic acid because such sources do not indicate a role for the nucleic acid or its uses. Indeed, there is often no knowledge of the sequences present in such sources until their presence is hypothesized as a result of using hindsight in light of a new sequence.

The identity of a protein or nucleic acid sequence is frequently established based on a sequence alignment of the DNA, RNA, or amino acids. Multiple alignments of such sequences are important tools in studying biomolecules. The basic information they provide is identification of conserved sequence regions. This is very useful in designing experiments to test and modify the function of specific proteins, in predicting the function and structure of proteins, and in identifying new members of protein families. Sequences can be aligned across their entire length (global alignment) or only in certain regions (local alignment). This is true for pairwise and multiple alignments. Global alignments with respect to polynucleic acids or polypeptides usually need to use gaps (representing insertions/deletions) while local alignments can usually avoid them by aligning regions between gaps. In a sequence alignment, letters arranged over one another are called matched. If two matched letters are equal, the match is called an identity otherwise the match is called a substitution or mismatch. An insertion or deletion (indel) is one or more letters aligned against a gap (−) and is considered the same as a mismatch for percent identity purposes (Waterman, M. S. 1995). In some cases a determination of the percent identity of a sequence relative to another sequence may be required. In such cases, the percent identity is measured in terms of the number of residues that are compared, as is customary in these arts.

Method to Identify Insulator/Enhancer-Blocking Sites for Use in Gene Therapy Vectors to Block Activation of Resident Chromosomal Genes.

In this application, an insulator element is defined as a DNA sequence that will block transcriptional regulatory sequences in a transgenic construct from acting on neighboring genes after the integration of the transgenic construct into a chromosome. A transgenic construct has an exogenous nucleic acid that for introduction into a host.

Herein is described a method for locating elements for testing, as described above. Persons of ordinary skill that follow these procedures will be able to rapidly identify a large number of insulator elements. Such elements, in combination with the many insulator elements already described in detail, provide a person of ordinary skill in the art a useful variety of insulator elements that may be used to practice the full scope of inventions taught herein.

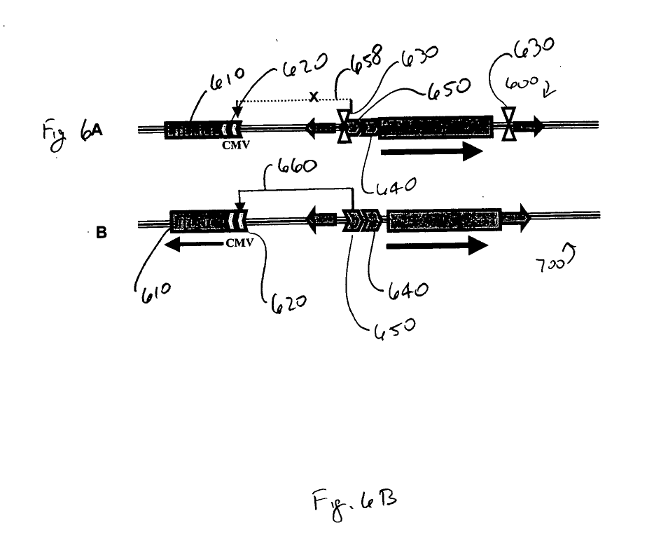

As described above, the 1.2 kb DNA sequence from the 5′ end of the chicken□-globin gene (5′ HS4) has been described as an insulator²³. The activity of the full-length HS4 insulator is found within a 250 bp “core” element²². There are two independent functions of the HS4 insulator- the blocking of enhancer activation and the blocking of chromosomal position effects¹²⁰. The enhancer blocking function of the HS4 insulator requires CTCF-binding protein⁸. The binding sites for CTCF-binding protein in the chicken β-globin loci are conserved in both mice and humans^{4.1}. The prominent structures of the human β-globin CTCF-enhancer-blocking motifs with respect to a transcriptional unit on a chromosome are shown in FIG. 5. There are two features to note to the 5′- and 3′ insulators (5-Ins and 3′-Ins respectively in the figure, labeled 500 and 510, respectively); first the 3′ sequence is an inverted form of the 5′ sequence; second, the sequences vary slightly. The transcriptional unit 520 is an exogenous nucleic acid sequence, e.g., a marker gene or therapeutic gene, operably linked to an enhancer and/or promoter.

Certain criteria are indicated herein. A person of ordinary skill in these arts, after reviewing these criteria, will be able to alter them as needed to suit a particular circumstance tailored to a particular research project. The indicated criteria for insulator elements are the following:

1) At least 15 basepairs of the 16 basepair human 5′- HS4-CTCF-binding site and 15 basepairs of the 16 basepair human 3′- HS1-CTCF-binding site or the mouse 3′-HS 1-CTCF-binding site must match in a pair of flanking insulators around a therapeutic gene. The mouse 3′-CTCF-binding site works as well or better than the human sequence⁴¹ and is therefore included in the searching process. These sequences can be identified by using NIH BLAST algorithms (see Table 1). The sequences can be prioritized by on the basis of matching of 5 basepairs on either side of the human HS4-CTCF binding sites. Over 100 exact matches of the CTCF-binding sites found near the human and or mouse β-globin loci exist in the human genome, and there are likely many more functional CTCF-binding sites that are near matches since the CTCF-binding sites from the chicken, mouse, and human β-globin sites have some sequence flexibility while maintaining function across species. The human genomic sequences that are predicted to contain CTCF-binding sites can be placed into test vectors by themselves or in conjunction with other CTCF- or other insulator-binding sites to test for the level of insulation provided.

2) The motif binds to CTCF or equivalent polypeptide. The test for this function is mobility shift assay similar to that employed to define transposase activity, and described in detail in the literature^{159,160}.

3) The motif has functional activity in blocking enhancer activation as defined by inhibiting enhancer activation of a promoter that is separated from an enhancer by the insulator element. This test is described in the following section.

TABLE I

Examples of Human sequences Matching CTCF-binding sites

| | | |
|---|---|---|
| GeneBank # | Chromosome | DNA sequence |

MATCHES TO THE HUMAN 5′ HS5 CTCF-binding site (NG_00007.3)

| | | | |
|---|---|---|---|
| NG_000007.3 | Chrm 11 | ttatga CCACTAGAGGGAAGAA gatacc | SEQ ID NO: 1 |
| | | \| \|\|\|\|\|\|\|\|\|\|\|\|\|\|\|\| \| | |
| AC002040 | Chrm 16 | gtcaag CCACTAGAGGGAAGAA aacttt | SEQ ID NO: 2 |
| | | \| \|\|\|\|\|\|\|\|\|\|\|\|\|\|\|\| \| \| | |
| AC068492 | Chrm 2 | ccccgc CCACTAGAGGGAAGAA aaaaaa | SEQ ID NO: 3 |
| | | \| \|\|\|\| \|\|\|\|\|\|\|\|\|\|\| \|\|\|\| \|\| \| | |
| AL159163.40 | Chrm. 6 | tcatga CCACTAGAGGGCAGAA gagaaa | SEQ ID NO: 4 |
| | | \| \|\|\|\| \| \|\|\|\|\|\|\|\|\|\|\|\|\|\| \|\| | |
| AP003049.2 | Chrm. 11 | tgatga CTACTAGAGGGAAGAA gaaggg | SEQ ID NO: 5 |

MATCHES TO THE HUMAN 3′ HS1 CTCF-BTNDING SITE (X54282).

| | | | |
|---|---|---|---|
| X54282 | Chrm. 11 | aactac TTCTGACCCCTAGTGG tgtcca | SEQ ID NO: 6 |
| | | \|\| \|\|\|\|\|\|\|\|\|\|\|\|\|\|\|\| | |
| AC067801 | Chrm. 18 | gtgtaa TTCTGACCCCTAGTGG ctgagg | SEQ ID NO: 7 |
| | | \|\| \|\|\|\|\|\|\|\|\|\|\|\|\|\|\| \| \| | |
| AC100821 | Chrm. 8 | tcaaac TTCTGACCCCTAGTGA tccacc | SEQ ID NO: 8 |
| | | \|\|\| \|\|\|\|\|\|\|\|\|\|\|\|\|\| \| \| \| | |
| AC135950 | Chrm. 16 | gtatac TTCTGACCCCTAGTAG gataaa | SEQ ID NO: 9 |
| | | \| \| \|\|\|\|\|\|\|\|\|\|\|\|\|\|\| \| | |
| BX649553 | Chrm. X | ggatgc TTCTGACCCCTAGTGT ccaaaa | SEQ ID NO: 10 |

MATCHES TO THE MOUSE 3′ HS1 CTCF-BTNDING SITE (AF133300.2)

| | | | |
|---|---|---|---|
| AF133300.2 | Chrm. 7 | tgcata CCAGTAGGGGGCAGAA gtgttc | SEQ ID NO: 11 |
| | (mus) | \| \| \|\|\|\|\|\|\|\|\|\|\|\|\|\|\|\| \|\|\|\| \| | |
| AC073065.6 | Chrm. 2 | tcactg CCAGTAGGGGGCAGAA gtgtac | SEQ ID NO: 12 |
| | | \|\|\|\|\|\|\|\|\|\|\|\|\|\|\|\| \|\|\| | |
| AL109955 | Chrm. 20 | atgcag CCAGTAGGGGGCAGAA gtgggg | SEQ ID NO: 13 |
| | | \| \|\|\|\|\|\|\|\|\|\|\|\|\|\|\|\| \| \| \| | |
| NG_000002.1 | Chrm.22 | gagaat CCAGTAGGGGGCAGAA gagacc | SEQ ID NO: 14 |
| | | \| \|\|\|\|\|\|\|\|\|\|\|\|\|\|\|\| \| \| | |
| AC123023.4 | Chrm. 3 | gctttg CCAGTAGGGGGCAGAA gagggt | SEQ ID NO: 15 |

NOTE:
flanking sequences in lower case; reference CTCF-binding sites shown in bold upper case:
CCACTAGAGGGAAGAA is SEQ ID NO: 16;
TTCTGACCCCTAGTGG is SEQ ID NO: 17;
CCAGTAGGGGGCAGAA is SEQ ID NO: 18.

Testing of Insulator Elements in Transposon

Persons of ordinary skill in these arts may use this protocol to determine if a nucleic acid sequence is an insulator element. Putative insulator elements are added immediately inside both ends of a transposon vector to keep the transgenic enhancers from activating neighboring genes using standard recombinant DNA techniques that have been used in the past for scs/scs′ and A-element insulators, and which are described in detail in the literatures^{19,20}. The assay involves measuring expression from the luciferase (ffLUC) gene, under the direction of the CMV minimal promoter, as a result of enhancer activity from within a transposon vector with and without border elements.

The strategy is shown in FIG. 6, which represents the features of a test plasmid that will be transfected into HeLa cells. Transposon plasmids 600, 700 contain the firefly luc gene 610 with a minimal CMV promoter 620 outside a Sleeping Beauty transposon. Putative insulators 630 are placed as shown, with control 700 not receiving an insulator element. Both transposons 600, 700 contain two species of luciferase reporter gene 610, and a CAGS promoter 640. The dotted line 658 with an “X” in the top panel indicates that enhancer activity from the transgenic enhancer 650 is blocked from activating the minimal CMV promoter 620 for transposon 600 but not for transposon 700, which lacks insulator element 630. The enhancer 650 is able to activate the CMV promoter when insulators are lacking, as shown by the solid line-arrow 660 in FIG. 6B.

Following transfection, with either standard sleeping beauty transposons (FIG. 6B) or Activation-Safe (A-S) transposons (FIG. 6A) with a Renilla (ren) luciferase reporter genea¹⁶¹ in the transposon, transient expression of firefly (ff) luciferase and Renilla luciferase is measured 48 hrs after transfection. The ratio of ffLUC/renLUC will indicate the level of activation of the firefly luc gene by the CAGGS promoter. If the putative sequence is an insulator, one will not observe ffLUC expression in the absence of an enhancer for the minimal CMV promoter. As a control, one deletes the CAGGS promoter from both the A-S transposons and from the Sleeping Beauty transposons. This provides the background level of luc expression that will be subtracted in the experiments using complete transposons with and without insulators. In this case one should not observe significant expression of either ffLUC or renLUC. One expects that the A-S transposons will exhibit ffLUC/renLUC ratios much lower than those from Sleeping Beauty transposons.

References

Textual citations to numbered references are found in the numbered list below; citations by author name are found in the list arranged alphabetically by author names.

1. Amsterdam, A., S. Lin, and N. Hopkins 1995. The Aequorea Victoria green fluorescent protein can be used as a reporter in live zebrafish embryos. Dev Biol 171: 123-129.

2. Amsterdam, A., S. Lin, L. G. Moss, and N. Hopkins 1996. Requirements for green fluorescent protein detection in transgenic zebrafish embryos. Gene 173:99-103.

3. Arai, Y., S. Kajihara, J. Masuda, S. Ohishi, K. Zen, J. Ogata, and T. Mukai 1994. Position-independent, high-level, and correct regional expression of the rat aldolase C gene in the central nervous system of transgenic mice. Eur J Biochem 221: 253-260.

4. Akasaka, K., A. Nishimura, K. Takata, K. Mitsunaga, F. Mibuka, H. Ueda, S. Hirose, K. Tsutsui and H. Shimada 1999. Upstream element of the sea urchini arylsulfatase gene serves as an insulator. Cell Mol Biol 45: 555-565.

5. Barges, S., J. Mihaly, M. Galloni, K. Hastrom, M. Muller, G. Shanower, P. Schedl, H. Gyurkovics and F. Karch 2000. The Fab-8 boundary defines the distal limit of the bithorax complex iab-7 domain and insulates iab-7 from initiation elements and a PRE in the adjacent iab-8 domain. Development 127: 779-790.

6. Belgrader, P., R. Dey, and R. Berezney 1991. Molecular cloning of matrin 3. A 125-kilodalton protein of the nuclear matrix contains an extensive acidic domain. J Biol Chem 266:9893-9.

7. Belgrader, P., A. J. Siegel, and R. Berezney 1991. A comprehensive study on the isolation and characterization of the HeLa S3 nuclear matrix. J Cell Sci 98: 281-291.

8. Bell, A. C., A. G. West and G. Felsenfeld 1999. The protein CTCF is required for the enhancer blocking activity of vertebrate insulators. Cell 98: 387-396.

9. Bell, A. C., A. G. West and G. Felsenfeld 2001. Insulators and boundaries: versatile regulatory elements in the eukaryotic genome. Science 291: 447-450.

10. Berrios, M., N. Osheroff, and P. A. Fisher 1985. In situ localization of DNA topoisomerase II, a major polypeptide component of the Drosophila nuclear matrix fraction. Proc Natl Acad Sci USA 82: 4142-4126.

11. Bode, J., and K. Maass 1988. Chromatin domain surrounding the human interferon-beta gene as defined by scaffold-attached regions. Biochemistry 27: 4706-11.

12. Bonifer, C., A. Hecht, H. Saueressig, D. Winter, A. Sippel 1991. Dynamic chromatin: the regulatory domain organization of eukaryotic gene loci. J Cell Biochem 47: 99-108.

13. Bonifer, C., M. C. Huber, N. Faust, and A. E. Sippel 1996. Regulation of the chicken lysozyme locus in transgenic mice. Crit Rev Eukaryot Gene Expr 6: 285-297.

14. Bonifer, C., M. C. Huber, U. Jagle, N. Faust, and A. E. Sippel 1996. Prerequisites for tissue specific and position independent expression of a gene locus in transgenic mice. J Mol Med 74: 663-671.

15. Bonifer, C., M. Vidal, G. Grosveld and A. E. Sippel 1990. Tissue specific and position independent expression of the complete gene domain for chicken lysozyme in transgenic mice. EMBO J. 9: 2843-2848.

16. Bonifer, C., N. Yannoutsos, G. Kruger, F. Grosveld, and A. E. Sippel 1994. Dissection of the locus control function located on the chicken lysozyme gene domain in transgenic mice. Nucleic Acids Res 22: 4202-4210.

17. Boulikas, T. 1995. Chromatin domains and prediction of MAR sequences. Intl Rev Cytol 162A: 279-388.

18. Cai, H., and M. Levine. 1995. Modulation of enhancer-promoter interactions by insulators in the Drosophila embryo. Nature 376: 533-536.

18a. Cai, H. and P. Shen 2001. Effects of cis arrangement of chromatin insulators on enhancer-blocking activity. Science. 291: 493-495.

19. Caldovic, L., D. Agalliu, and P. B. Hackett 1999. Position-independent expression of transgenes in zebrafish. Transgenic Res. 8: 321-334.

20. Caldovic, L. and P. B. Hackett 1995. Development of position-independent expression vectors and their transfer into transgenic fish. Mol Mar Biol Biotechnol 4: 51-61.

21. Cavazzana-Calvo, M., S. Hacein-Bey, G. de Saint Basile, F. Gross, E. Yvon, P. Nusbaum, et al. 2000. Gene therapy of human severe combined immunodeficiency (SCID)-X1 disease. Science. 288: 669-672.

22. Chung, J. H., A. C. Bell and G. Felsenfeld 1997. Characterization of the chicken β-globin insulator. Proc Natl Acad Sci USA 94: 575-580.

23. Chung, J. H., M. Whiteley, and G. Felsenfeld 1993. A 5′ element of the chicken beta-globin domain serves as an insulator in human erythroid cells and protects against position effect in Drosophila. Cell 74: 505-514.

24. Cockerill, P. N. 1990. Nuclear matrix attachment occurs in several regions of the IgH locus. Nucleic Acids Res 18: 2643-2648.

25. Cockerill, P. N., and W. T. Garrard. 1986. Chromosomal loop anchorage of the kappa immunoglobulin gene occurs next to the enhancer in a region containing topoisomerase II sites. Cell 44:273-82.

26. Cockerill, P. N., and W. T. Garrard. 1986. Chromosomal loop anchorage sites appear to be evolutionarily conserved. FEBS Lett 204: 5-7.

27. Cockerill, P. N., M. H. Yuen, and W. T. Garrard 1987. The enhancer of the immunoglobulin heavy chain locus is flanked by presumptive chromosomal loop anchorage elements. J Biol Chem 262: 5394-5397.

28. Cook, P. R. 1995. A chromomeric model for nuclear and chromosome structure. J Cell Sci 108: 2927-2935.

29. Cunningham, J. M., M. E. Purucker, S. M. Jane, B. Safer, E. F. Vanin, P. A. Ney, C. H. Lowrey, and A. W. Nienhuis 1994. The regulatory element 3′ to the α gamma-globin gene binds to the nuclear matrix and interacts with special A-T-rich binding protein 1 (SATB1), an SAR/MAR-associating region DNA binding protein. Blood 84: 1298-1308.

30. Culp, P., C. Nusslein-Volhard, and N. Hopkins 1991. High-frequency germ-line transmission of plasmid DNA sequences injected into fertilized zebrafish eggs. Proc Natl Acad Sci USA 88: 7953-7957.

31. Darnell, J., H. Lodish and D. Baltimore 1990. Molecular Cell Biology. Scientific American Books. W.h. Freeman and Co. New York. Pp1104.

32. Davie, J. R. 1995. The nuclear matrix and the regulation of chromatin organization and function. Intl Rev Cytol 162A: 191-250.

33. Davie, J. R. 1996. Histone modifications, chromatin structure, and the nuclear matrix. J Cell Biochem 62: 149-557.

34. Dickinson, L. A., T. Joh, Y. Kohwi, and T. Kohwi-Shigematsu 1992. A tissue-specific MAR/SAR DNA-binding protein with unusual binding site recognition. Cell 70: 631-645.

35. Dillon, N. and F. Grossveld 1993. Transcriptional regulation of multigene loci: multilevel control. Trends Genet 9: 134-137.

36. DiSimone, P., A. DiLeonardo, G. Costanzo, R. Melfi and G. Spinnelli 2001. The sea urchin sns insulator blocks CMV enhancer following integration in human cells. Biochem Biophys Res Comm 284: 987-992.

37. Dunaway, M., J. Y. Hwang, M. Xiong, and H.-L. Yuen 1997. The activity of the scs and scs′ insulator elements is not dependent on chromosomal context. Mol Cell Biol 17: 182-189.

38. Eberharter, A., A. Grabher, G. Gstraunthaler, and P. Loidl 1993. Nuclear matrix of the lower eukaryote Physarum polycerphalum and the mammalian epithelial LLC-PK1 cell line. A comprehensive investigation of different preparation procedures. Eur J Biochem 212: 573-580.

39. Eissenberg, J. C., S. C. R. Elgin, and R. Paro 1995. Epigenetic regulation in Drosophila: a conspiracy of silence, p. 147-171. In S. C. R. Elgin (ed.), Chromatin Structure and Gene Expression. Oxford University Press, Oxford.

40. Ellis, J. and D. Pannell 2001. The β-globin locus control region versus gene therapy vectors: A struggle for expression. Clin. Genet 59: 17-24.

41. Farrell, C. M., West , A. G. and G. Felsenfeld 2002. Conserved CTCF insulator elements flank the mouse and human beta-globin loci. Mol Cell Biol. 22: 3820-3831.

42. Filippova, G. N., C. P. Thienes, B. H. Penn, D. H. Cho, Y. J. Hu, J. M. Moore, T. R. Klesert. V. V. Lobanenkov and S. J. Tapscott 2001. CTCF-binding sites flank CTG/CAG repeats and form a methylation-sensitive insulator at the DM1 locus. Nature Genet 28: 335-343.

43. Forrester, W. C., C. van Genderen, T. Jenuwein, and R. Groschedl 1994. Dependence of enhancer-mediated transcription of the immunoglobulin m gene on nuclear matrix attachment regions. Science 265: 1221-1227.

44. Forrester, W. C., L. A. Fernandez and R. Grosschedl 1999. Nuclear matrix attachment regions antagonize methylation-dependent repression of long-range enhancer-promoter interactions. Genes Dev 13: 3003-3014.

45. Gause, M, P. Morcillo and D, Dorsett 2001. Insulation of enhancer-promoter communication by a gypsy transposon insert in the Drosophila cut gene: Cooperation between suppressor of hairy-wing and modifier of mdg4 proteins. Mol Cell Biol 21: 4807-4817.

46. Gazner, M., J. Vazquez and P. Schedl 1999. The Zw5 protein, a component of the scs chromatin domain boundary, is able to block enhancer-promoter interaction. Genes Dev 13: 2098-2107.

47. Gdula, D. A., T. I. Gerasimova, and V. G. Corces 1996. Genetic and molecular analysis of the gypsy chromatin insulator of Drosophila. Proc Natl Acad Sci USA 93: 9378-9383.

48. Gerasimova, T. I., K. Byrd, and V. G. Corces 2000. A chromatin insulator determines the nuclear localization of DNA. Mol Cell 6: 1025-1035.

49. Gerasimova, T. I., D. A. Gdula, D. V. Gerasimov, O. Simonova, and V. G. Corces 1995. A Drosophila protein that imparts directionality on a chromatin insulator is an enhancer of position-effect variegation. Cell 82: 587-597.

50. Geyer, P. K. 1997. The role of insulator elements in defining domains of gene expression. Curr Op Genet & Devel 7: 242-248.

51. Ghosh, D. T. I. Gerasimova and V. G. Corces 2001. Interactions between the Su[Hw] and Mod(mdg4) protein required for gypsy insulator function. EMBO J 20: 2518-2527.

52. Gibbs, P. D., A. Peek, and G. Thorgaard 1994. An in vivo screen for the luciferase transgene in zebrafish. Mol Mar Biol Biotechnol 3: 307-316.

53. Gottschling, D. E., O. M. Aparicio, P. D. Zierath, B. L. Billington, and V. A. Zakian 1990. Position effect at S. cervisiae telomeres: reversible repression of Pol II transcription. Cell 63: 751-762.

54. Greer, P., V. Maltby, J. Rossant, A. Bernstein, and T. Pawson 1990. Myeloid expression of the human c-fps/fes proto-oncogene in transgenic mice. Mol Cell Biol 10: 2521-2527.

55. Hacein-Bey-Abina, S., F. Le Deist, F. Carlier, C. Bouneaud, C. Hue, J. P. De Villartay, et al. 2002. Sustained correction of X-linked severe combined immunodeficiency by ex vivo gene therapy. New Eng. J. Med. 346: 1185-1193.

56. Hackett, P. B., Z. Izsvak, Z. Ivics, and L. Caldovic 1999. Development of genetic tools for transgenic animals. IN Transgenic Animals in Agriculture. CAB International, Wallingford, UK. 19-35.

57. Hagstrom, K., M. Muller and P. Schedl 1996. Fab-7 functions as a chromatin domain boundary to ensure proper segment specification by the Drosophila bithorax complex. Genes Dev 10: 3202-3215.

58. Hakes, D. J., and R. Berezney 1991. Molecular cloning of matrin F/G: A DNA binding protein of the nuclear matrix that contains putative zinc finger motifs. Proc Natl Acad Sci USA 88: 6186-6190.

59. Hark, A. T., C. J. Schoenherr, D. J. Katz, R. S. Ingram, J. M. Levorse and S. M. Tilghman 2000. CTCF mediates methylation-sensitive enhancer-blocking activity at the H19/Igf2 locus. Nature 405: 486-489.

60. Harrison, D. A., D. A. Gdula, R. S. Coyne, and V. G. Corces 1993. A leucine zipper domain of the suppressor of Hairy-wing protein mediates its repressive effect on enhancer function. Genes Dev 7: 1966-1978.

61. Hart, C. M., K. Zhao, and U. K. Laemmli 1997. The scs′ boundary element: characterization of boundary element-associated factors. Mol Cell Biol 17: 999-1009.

62. Hastie, N. D. and J. O. Bishop 1976. The expression of three abundance classes of messenger RNA in mouse tissues. Cell 9: 761-764.

63. Holmgren, C., C. Kanduri, G. Dell, A. Ward, R. Mukhopadhya, M. Kanduri, V. Lobanenkov and R. Ohlsson 2001. CpG methylation regulates the Ifg2/H19 insulator. CurrBiol. 11: 1128-1130.

64. Hozak, P., A. M. Sasseville, Y. Raymond, and P. R. Cook 1995. Lamin proteins form an internal nucleoskeleton as well as a peripheral lamina in human cells. J Cell Sci 108: 635-644.

65. Hughes, T. A., A. Pombo, J. McManus, P. Hozak, D. A. Jackson, and P. R. Cook 1995. On the structure of replication and transcription factories. J Cell Sci 19: 59-65.

66. Inoue, T., H. Yamaza, Y. Sakai, S. Mizuno, M. Ohno, N. Hamasaki and Y. Fukumaki 1999. Position-independent human β-globin gene expression mediatd by a recombinant Adeno-associated virus vector carrying the chicken β-globin insulator. J Hum Genet 44: 152-162.

67. Jack, J., D. Dorsett, Y. Delotto, and S. Liu 1991. Expression of the cut locus in the Drosophila wing margin is required for cell type specification and is regulated by a distant enhancer. Development 113: 735-747.

68. Jackson, D. A., and P. R. Cook 1985. A general method for preparing chromatin containing intact DNA. EMBO J 4: 913-918.

69. Jackson, D. A., and P. R. Cook 1985. Transcription occurs at a nucleoskeleton. EMBO J 4: 919-925.

70. Jackson, D. A., and P. R. Cook 1995. The structural basis of nuclear function. Intl Rev Cytol 162A: 125-149.

71. Jackson, D. A., A. B. Hassan, R. J. Errington, and P. R. Cook 1993. Visualization of focal sites of transcription within human nuclei. EMBO J 12: 1059-1065.

72. Jackson, D. A., J. Yuan, and P. R. Cook 1988. A gentle method for preparing cyto- and nucleo-skeletons and associated chromatin. J Cell Sci 90: 365-378.

73. Jaenisch, R. 1988. Transgenic animals. Science 240:1468-1474.

74. Kalos, M. and R. E. Fournier 1995. Position-independent transgene expression mediated by boundary elements from the apolipoprotein B chromatin domain. Mol Cell Biol 15: 198-207.

75. Kanduri, C., V. Pant, D. Loukinov, E. Pugacheva, C. F. Qi, A. Wolffe, R. Ohlsson and V. V. Lobanenkov 2000. Functional association of CTCF with the insulator upstream of the H19gene is parent of origin-specific and methylation-sensitive gene. Curr Biol 10: 853-856.

76. Kellum, R., and P. Schedl 1991. A position-effect assay for boundaries of higher order chromosomal domains. Cell 64: 941-950.

77. Kellum, R., and P. Schedl 1992. A group of scs elements function as domain boundaries in an enhancer-blocking assay. Mol Cell Biol 12: 2424-231.

78. Kim, J., B. Shen, C. Rosen, and D. Dorsett 1996. The DNA-binding and enhancer-blocking domains of the Drosophila suppresor of Hairy-wing protein. Mol Cell Biol 16: 3381-3392.

79. Kornberg, R. D. and Y. Lorch 1992. Chromatin structure and transcription. Ann Rev Cell Biol 8: 563-587.

80. Krebs, J. E. and M. Dunaway 1996. DNA length is a critical parameter for eukaryotic transcription in vivo. Mol Cell Biol 16: 5821-5829.

81. Labrador, M. and V. G. Corces 2002. Setting the boundaries of chromatin domains and nuclear organization. Cell 111: 151-154.

82. Laemmli, U. K., E. Kas, L. Poljak, and Y. Adachi 1992. Scaffold-associated regions: cis-acting determinants of chromatin structural loops and functional domains. Curr Opin Genet Dev 2: 275-285.

83. Levy-Wilson, B., and C. Fortier 1989. The limits of the DNase I-sensitive domain of the human apolipoprotein B gene coincide with the locations of chromosomal anchorage loops and define the 5′ and 3′ boundaries of the gene. J Biol Chem 264: 21196-21204.

84. Lewin, B. 1993. Genes V.5^{th} Edition John Wiley & Sons. New York

85. Li, Q. and G. Stamatoyannopoulos 1994. Hypersensitive site 5 of the human β locus control region functions as a chromatin insulator. Blood 84: 1399-1401.

86. Li, Q. , S. Harju and K. R. Peterson 1999. Locus control regions: coming of age at a decade plus. Trends Genet. 15: 403-408.

87. Lin, S., S. Yang, and N. Hopkins 1994. lacZ expression in germline transgenic zebrafish can be detected in living embryos. Dev Biol 161: 77-83.

88. Loc, P. V., and W. H. Stratling 1988. The matrix attachment regions of the chicken lysozyme gene co-map with the boundaries of the chromatin domain. EMBO J 7: 655-664.

89. Lu, L. and J. Tower 1997. A transcriptional insulator element, the su(HW) binding site, protects a chromosomal DNA, replication origin from position effects. Mol Cell Biol 17: 2202-2206.

90. Luderus, M. E., A. de Graaf, E. Mattia, J. L. den Blaauwen, M. A. Grande, L. de Jong, and R. van Driel 1992. Binding of matrix attachment regions to lamin B1. Cell 70: 949-959.

91. Luderus, M. E., J. L. den Blaauwen, O. J. de Smit, D. A. Compton, and R. van Driel 1994. Binding of matrix attachment regions to lamin polymers involves single-stranded regions and the minor groove. Mol Cell Biol 14: 6297-6305.

92. McKnight, R. A., A. Shamay, L. Sankaran, R. J. Wall, and L. Hennighausen 1992. Matrix-attachment regions can impart position-independent regulation of a tissue-specific gene in transgenic mice. Proc Natl Acad Sci USA 89: 6943-6947.

93. McKnight ,R. A., M. Spencer, R. J. Wall and L. Hennighausen 1996. Severe position effects imposed on a 1 kb mouse whey acidic protein gene promoter are overcome by heterologous matrix attachment regions. Mol Reprod Dev. 44: 179-184.

94. Melfi, R., F. Palla, P. Di Simone, C. Alessandro, L. Cali, L. Anello and G. Spinelli 2000. Functional characterization of the enhancer blocking element of the sea urchin early histone gene cluster reveals insulator properties and three essential cis-acting sequences. J Mol Biol 304: 753-763.

95. Michel, P. J., and R. Tijan 1989. Transcriptional regulation in mammalian cells by sequence-specific DNA binding proteins. Science 245: 371-378.

96. Mirkovitch, J., M. E. Mirault, and U. K. Laemmli 1984. Organization of the higher-order chromatin loop: specific DNA attachment sites on nuclear scaffold. Cell 39: 223-2232.

97. Miynarova, L., A. Loonen, J. Heldens, R. C. Jansen, P. Keizer, W. J. Stiekema, and J. P. Nap 1994. Reduced position effect in mature transgenic plants conferred by the chicken lysozyme matrix-associated region. Plant Cell 6: 417-426.

98. Modolell, J., W. Bender, and M. Meselson 1983. Drosophila melanogaster mutations suppressible by the suppressor of Hairy-wing are insertions of a 7.3-kilobase mobile element. Proc Natl Acad Sci USA 80: 1678-1682.

99. Mongelard, F. and V. G. Corces 2001. Two insulators are not better than one. Nature Struct. Biol. 8: 192-194.

100. Morris, J. R., J.-L. Chen, P. K. Geyer, and C.-T. Wu 1998. Two modes of transvection: Enhancer action in trans and bypass of a chromatin insulator in cis. Proc Natl Acad Sci USA 95: 10740-10745.

101. Muravyova, E., A. Golovnin, E. Gracheva, A. Parshikov, T. Belenkaya, V. Pirrotta, and P. Georgiev 2001. Loss of insulator activity by paired Su(Hw) chromatin insulators. Science 291: 495-498.

102. Mutskov. V. J., C. M. Farrell, P. A. Wade, A. P. Wolffe, and G. Felsenfeld 2002. The barrier function of an insulator couples high histone acetylation levels with specific protection of promoter DNA from methylation. Genes Dev 16: 1540-1554.

103. Nakagomi, K., Y. Kohwi, L. A. Dickinson, and T. Kohwi-Shigematsu 1994. A novel DNA-binding motif in the nuclear matrix attachment DNA-binding protein SATB1. Mol Cell Biol 14:1852-1860.

104. Nakayasu, H., and R. Berezney 1991. Nuclear matrins: identification of the major nuclear matrix proteins. Proc Natl Acad Sci USA 88: 10312-10316.

105. Namciu, S. J., K. B. Blochlinger, and R. E. K. Fournier 1998. Human matrix attachment regions insulate transgene expression from chromosomal position effects in Drosphila melanogaster. Mol Cell Biol 18: 2382-2391.

106. Oki, M. and R. T. Kamakaka 2002. Blockers and barriers to transcription: competing activities? Curr Opin Cell Biol 14: 299-304.

107. Palla, F., R. Melfi, L. Anello, M. DiBernardo, and G. Spinelli 1997. Enhancer blocking activity located near the 3′ end of the sea urchin early H2A histone gene. Proc Natl Acad Sci USA. 94: 2272-2277

108. Palmiter, R. D., E. P. Sandgren, D. M. Koeller, and R. L. Brinster 1993. Distal regulatory elements from the mouse metallothionein locus stimulate gene expression in transgenic mice. Mol Cell Biol 13: 5266-5275.

109. Pannell, D. and J. Ellis 2001. Silencing of gene expression: implications for design of retrovirus vectors. Rev MedVirol 11: 205-217.

110. Parkhurst, S. M., D. A. Harrison, M. P. Remington, C. Spana, R. L. Kelley, R. S. Coyne, and V. G. Corces 1988. The Drosophila su(Hw) gene, which controls the phenotypic effect of the gypsy transposable element, encodes a putative DNA-binding protein. Genes Dev 2: 1205-1215.

111. Peifer, M., and W. Bender 1986. The anterobithorax and bithorax mutations of the bithorax complex. EMBO J 5: 2293-2303.

112. Peifer, M., and W. Bender 1988. Sequences of the gypsy transposon of Drosophila necessary for its effects on adjacent genes. Proc Natl Acad Sci USA 85: 9650-9654.

113. Phi-Van, L., J. P. von Kries, W. Ostertag, and W. H. Stratling 1990. The chicken lysozyme 5′ matrix attachment region increases transcription from a heterologous promoter in heterologous cells and dampens position effects on the expression of transfected genes. Mol Cell Biol 10: 2302-2307.

114. Piental, K. J., R. H. Getzenberg and D. S. Cofey 1991. Cell structure and DNA organization. CRC Criti Rev Euk Gene Exp 1: 355-385.

115. Pikaart, M. J., F. Recillas-Targa, and G. Felsenfeld 1998. Loss of transcriptional activity of a transgene is accompanied by DNA methylation and histone deacetylation and is prevented by insulators. Genes Dev 12: 2852-2862.

116. Pillus, L., and M. Grunstein 1995. Chromatin structure and epigenetic regulation in yeast, p. 123-146. In S. G. R. Elgin (ed.), Chromatin Structure and Gene Expression. Oxford University Press, Oxford.

117. Pombo, A., and P. R. Cook 1996. The localization of sites containing nascent RNA and splicing factors. Exp Cell Res 229: 201-203.

118. Porter, S. D., J. Hu and C. B. Gilka 1999. Distal upstream tyrosinase S/MAR-containing sequences has regulatory properties specific to subsets of melanocytes. Dev Genet 25: 40-48.

119. Prioleau, M. N., P. Nony, M. Simpson and G. Felsenfeld 1999. An insulator element and condensed chromatin region separate the chicken b-globin locus from an independent regulted erythroid-specific folate receptor gene. EMBO J. 18: 4035-4048.

120. Recillas-Targa, F., A. C. Bell, and G. Felsenfeld 1999. Positional enhancer-blocking activity of the chicken beta-globin insulator in transiently transfected cells. Proc Natl Acad Sci. USA 96: 14354-14359.

121. Renauld, H., O. M. Aparicio, P. D. Zierath, B. L. Billington, S. K. Chablani, and D. E. Gottchling 1993. Silent domains are assembled continuously from the telomere and are defined by promoter distance and strength, and SIR3 dosage. Genes Dev 7: 1133-1145.

122. Reuter, G., and I. Wolff 1981. Isolation of dominant suppressor mutations for position-effect variegation in Drosophila melanogaster. Mol Gen Genet 182: 516-519.

123. Richards, E. J. and S. C. R. Elgin 2002. Epigenetic codes for heterochromatin formation and silencing: rounding up the usual suspects. Cell 108: 489-500.

124. Rivella, S., J. A. Callegari, C. May, C. W. Tan and M. Sadelain 2000. The cHS4 insulator increases the probability of retroviral expression at random chromosomal integration sites. J Virol 74: 4679-4687.

125. Romig, H., F. O. Fackelmayer, A. Renz, U. Ramsperger, and A. Richter 1992. Characterization of SAF-A, a novel nuclear DNA binding protein from HeLa cells with high affinity for nuclear matrix/scaffold attachment DNA elements. EMBO J 11: 3431-3440.

126. Roseman, R. R., V. Pirrotta, and P. K. Geyer. 1993. The su(Hw) protein insulates expression of the Drosophila melanogaster white gene from chromosomal position-effects. EMBO J 12: 435-442.

127. Schedl, P., and F. Grosveld 1995. Domains and boundaries, p. 172-196. In E. SCR. (ed.), Chromatin Structure and Gene expression. Oxford University Press, Oxford.

128. Scott, K. S., and P. K. Geyer 1995. Effects of the su(Hw) insulator protein on the expression of the divergently transcribed Drosophila yolk protein genes. EMBO J 14: 6258-6267.

129. Singer, R. H. and M. R. Green 1997. Compartmentalization of eukaryotic gene expression: causes and effects. Cell 91: 291-294.

130. Sepich, D. Personal communication.

131. Smith, P. A., and V. G. Corces 1992. The suppressor of Hairy-wing binding region is required for gypsy mutagenesis. Mol Gen Genet 233: 65-70.

132. Snustad, D. P., M. J. Simmons and J. B. Jenkins 1997. Principles of Genetics, 2^{nd} edition. John Wiley and Sons. New York. pp. 864

133. Stief, A., D. M. Winter, W. H. Stratling, and A. E. Sippel. 1989. A nuclear DNA attachment element mediates elevated and position-independent gene activity. Nature 341:343-5.

134. Strahl, B. D. and C. A. Allis 2000. The language of covalent histone modifications. Nature 403: 41-45.

135. Steinwaerder, D. S. and A. Lieber 2000. Insulation from viral transcriptional regulatory elements improves inducible transgene expression from adenovirus vectors in vitro and in vivo. Gene Ther 7: 556-567.

136. Stuart, G. W., J. V. McMurray, and M. Westerfield. 1988. Replication, integration and stable germ-line transmission of foreign sequences injected into early zebrafish embryos. Development 103:403-12.

137. Stuart, G. W., J. R. Vielkind, J. V. McMurray, and M. Westerfield. 1990. Stable lines of transgenic zebrafish exhibit reproducible patterns of transgene expression. Development 109:577-84.

138. Thorey, I. S., G. Cecena, W. Reynolds, and R. G. Oshima. 1993. Alu sequence involvement in transcriptional insulation of the keratin 18 gene in transgenic mice. Mol Cell Biol 13:6742-51.

139. Thompson, E. M., E. Christians, M. G. Stinnakre, and J. P. Renard. 1994. Scaffold attachment regions stimulate HSP70.1 expression in mouse preimplantation embryos but not in differentiated tissues. Mol Cell Biol 14:4694-703.

140. Tilghman, S. M., and W. F. Huntington. 1995. Epigenetic regulation in mammals, p. 197-222. In S. C. R. Elgin (ed.), Chromatin Structure and Gene Expression. Oxfprd University Press, Oxford.

141. Udvardy, A., E. Maine, and P. Schedl. 1985. The 87A7 chromomere. Identification of novel chromatin structures flanking the heat shock locus that may define the boundaries of higher order domains. J Mol Biol 185:341-58.

142. Udvardy, A., and P. Schedl. 1991. Chromatin structure, not DNA sequence specificity, is the primary determinant of topoisomerase II sites of action in vivo. Mol Cell Biol 11:4973-84.

143. Udvardy, A., and P. Schedl. 1993. The dynamics of chromatin condensation: redistribution of topoisomerase II in the 87A7 heat shock locus during induction and recovery. Mol Cell Biol 13:7522-30.

144. von Kries, J. P., H. Buhlrmester, and W. H. Stratling. 1991. A matrix/scaffold attachment region binding protein: identification, purification, and mode of binding. Cell 64:123-35.

145. von Kries, J. P., O. Rosorius, H. Buhrmester, and W. H. Stratling. 1994. Biochemical properties of attachment region binding protein ARBP. FEBS Lett 342:185-8.

146. von Kries, J. P., F. Buck, and W. H. Stratling. 1994. Chicken MAR binding protein p120 is identical to human heterogeneous nuclear ribonucleoprotein (hnRNP) U. Nucleic Acids Res 22:1215-20.

147. Walters, M. C., S. Fiering, E. E. Bouhassira, D. Scalzo, S. Goeke, W. Magis, D. Garrick, E. Whitelaw and D. I. Martin 1999. The chicken beta-globin 5′HS4 boundary element blocks enhancer-mediated suppression of silencing. Mol Cell Biol 19: 3714-3726.

148. Weintraub, H., A. Larsen, and M. Groudine. 1981. Alpha-globin-gene switching during the development of chicken embryos: expression and chromosome structure. Cell 24:333-344.

149. Weitzel, J. M., H. Buhrmester, and W. H. Stratling 1997. Chicken MAR-binding protein ARBP is homologous to rat methyl-CpG-binding protein MeCP2. Mol Cell. Biol 17: 5656-5666.

150. West, A. G., M. Gaszner and G. Felsenfeld 2002. Insulators: many functions, many mechanisms. Genses Dev. 16: 271-278.

151. Wilson, C., H. J. Bellen, and W. J. Gehring. 1990. Position effects on eukaryotic gene expression. Annu Rev Cell Biol 6:679-714.

152. Wolffe, A. P. 1994. Insulating chromatin. Curr. Biol. 4: 85-87.

153. Woodcock, C. L., S. A. Grigoryev, R. A. Horowitz, and N. Whitaker. 1993. A chromatin folding model that incorporates linker variability generates fibers resembling the native structures. Proc Natl Acad Sci USA 90: 9021-9025.

154. Woodcock, C. L. 1994. Chromatin fibers observed in situ in frozen hydrated sections—native fiber diameter is not correlated with nucleosome repeat length. J Cell Biol 125:11-19.

155. Yu, J. J. H. Bock, J. L. Slightom and B. Villeponteau 1994. A 5′β-globin matrix-attachment region and polyoma enhancer together confer position-independent transcription. Gene 139: 139-145

156. Zhan, H. C., D. P. Liu and C. C. Liang 2001. Insulator: from chromatin domain boundary to gene regulation. Hum Genet 109: 471-478.

157. Zhao, K., C. M. Hart, and U. K. Laemmli. 1995. Visualization of chromosomal domains with boundary element-associated factor BEAF-32. Cell 81: 879-889.

158. Zhou, J., S. Barolo, P. Szymanski and M. Levine 1996. The Fab-7 element of the bithorax complex attenuates enhancer promoter interactions in the Drosophila embryo. Genes Dev 10: 31295-3201.

159. Ivics, Z., P. B. Hackett, R. H. Plasterk, and Zs. Izsvak (1997). Molecular reconstruction of Sleeping Beauty, a Tc1-like transposon from fish, and its transposition in human cells. Cell 91: 501-510.

160. Hackett, P. B., Z. Ivics and Zs. Izsvak (2002). DNA-based transposon system for the introduction of nucleic acid into DNA of a cell. U.S. Pat. No. 6,489,458

161. Sharf, B. A. et al. (1996). Dual luciferase reporter assays: An advanced co-reporter technology integrating firefly and Renilla luciferase assays. Promega Notes 57:2-9.

162. Hodgkin, J. and R. K. Herman (1998). Changing styles in C. elegans genetics. Trends Genet. 14: 352-357.

163. Prak, E. T. L. and H. H. Kazazian Jr. (2000). Mobile elements and the human genome. Nature Genet. Rev. 1: 134-144.

164. Sharrat, D. J. (1995). Mobile Genetic Elements. IRL Press Oxford

165. Zhang, P. and A. C. Spradling (1994). Insertional mutagenesis of Drosophila heterochromatin with single P elements. Proc. Natl. Acad. Sci. USA91:3539-3543.

166. Morgan, B. A., F. L. Conlon, M. Manzanares, J. B. A. Millar, N. Kanuga, J. Sharpe, R. Krumlauf, J. C. Smith, and S. G. Sedgwick (1996). Transposon tools for recombinant DNA manipulation: Characterization of transcriptional regulators from yeast, Xenopus, and mouse. Proc. Natl. Acad. Sci. USA 93: 2801-2806.

167. Goryshin, I. Y., J. Jendrisak, L. M. Hoffman, R. Meis, and W. S. Reznikoff(2000). Insertional transposon mutagenesis by electroporation of released Tn5 transposition complexes. Nature Biotech. 18: 97-100.

168. Hoffman, L. M., J. J. Jendrisak, R. J. Meis, I. Y. Goryshin and S. W. Reznikof (2000). Transposon insertional mutagenesis and direct sequencing of microbial genomes. Genetica 108: 19-24.

169. Klinakis, A. G., L. Zagoraiou, D. K. Vassilatis and C. Savakis (2000). Genome-wide insertional mutagenesis in human cells by the Drosophila mobile element Minos. EMBO Reports 1: 416-421.

170. Akerley, B. J. E. J. Rubin, A. Camilli, D. J. Lampe, H. M. Robertson and J. J. Mekalanos (1998). Systematic identification of essential genes by in vitro mariner mutagenesis. Proc. Natl. Acad. Sci. USA 95: 8927-8932.

171. Zhang, G., V. Budker and J. A. Wolff (1999) High levels of foreign gene expression in hepatocytes after tail vein injections of naked plasmid DNA. Hum. Gene Ther. 10: 1735-1737.

172. Kren B. T., P. Bandyopadhyay and C. J. Steer (1998). In vivo site-directed mutagenesis of the factor IX gene by chimeric RNA/DNA oligonucleotides. Nature Medicine 4: 285-290.

Anderson, W. F. (19998). Human gene therapy. Nature 392 (suppl): 25-30.

Amanuma, K., H. Takeda, H. Amanuma and Y. Aoki (2000). Transgenic zebrafish for detecting hazardous mutations caused by compounds in aquatic environments. Nature Biotech. 18: 62-65.

Ausubel, F. et al. (1994). Current Protocols in Molecular Biology, Contents V. 1, 2, and 3. Table of Contents.

Bell, A. C., A. G. West and G. Felsenfeld (1999). The protein CTCF is required for the enhancer blocking activity of vertebrate insulators. Cell 98: 387-396.

Bell, A. C., A. G. West and G. Felsenfeld (2001). Insulators and boundaries: versatile regulatory elements in the eukaryotic genome. Science 291: 447-450.

Bestor, T. H. (1998). Methylation meets acetylation. Nature 393: 311-312.

Bird, A. (1997). Does DNA methylation control transposition of selfish elements in the germline? Trends Genet. 13: 469-470.

Bonifer, C., A. Hecht, H. Saueressig, D. M. Winter and A. E. Sippel (1991). Dynamic chromatin: the regulatory domain organization of eukaryotic gene loci. J. Cell. Biochem. 47: 99-108.

Borrelli, E., R. Heyman, M. Hsi, and R. M. Evans (1988). Targeting of an inducible toxic phenotype in animal cells. Proc. Natl. Acad. Sci. USA 85: 7572-7576.

Caldovic, L. and P. B. Hackett (1995). Development of position-independent expression vectors and their transfer into transgenic fish. Mol. Mar. Biol. Biotech. 4: 51-61.

Caldovic, L., D. Agalliu and P. B. Hackett (1999). Position-independent expression and germline transmission of transgenic DNA in zebrafish. Trans. Res. 8: 321-334.

Camper, S. A. (1987). Research applications of transgenic mice. BioTechniques 5: 638-650.

Carvan, M. J., et al. (2001). Oxidative stress in zebrafish cells: potential utility of transgenic zebrafish as a deployable sentinel for site hazard ranking. Sci. Total Environ. 274: 183-196.

Chung, J. H., A. C. Bell, G Felsenfeld (1997). Characterization of the chicken β-globin insulator. Proc. Natl. Acad. Sci. USA 94: 575-580.

Doefler, W. (1992). DNA methylation: eukaryotic defense against the transcription of foreign genes. Microbial. Pathogenesis 12: 1-8.

Dove, A. (2000). Milking the genome for profit. Nature Biotech. 18: 1045-1048.

Fahrenkrug, S. C., K. Clark, and P. B. Hackett (1999). Dicistronic gene expression in developing zebrafish. Mar. Biotech. 1: 552-561.

Farrell CM, West AG, Felsenfeld G.(2002). Conserved CTCF insulator elements flank the mouse and human beta-globin loci. Mol Cell Biol. 22: 3820-3831.

FDA (2002). See worldwide web at fda.gov/bbs/topics/ANSWERS/2003/ANS01190.html

Geyer, P. K. (1997) The role of insulator elements in defining domains of gene expression. Curr. Opin. Genet. Devel. 7: 242-248.

Hackett, P. B. and M. C. Alvarez (2000). The molecular genetics of transgenic fish. Recent Adv. Mar. Biotech. 4:77-145.

Hackett, P. B.,Z. Izsvak, Z. Ivics, and L. Caldovic (1999). Development of genetic tools for transgenic animals. IN Transgenic Animals in Agriculture. CAB International, Wallingford, UK. 19-35.

Ivics, Z., Z, Izsvak and P. B. Hackett (1999). Genetic applications of transposons and other repetitive elements in zebrafish. IN Detrich, H. W., M. Westerfield, and L. I. Zon (eds.) The Zebrafish: Genetics and Genomics Meth. Cell Biol. 60: 99-131.

Izsvak, Z., Z. Ivics, and P. B. Hackett (1997). Repetitive elements and their genetic applications in zebrafish. Biochem. Cell Biol. 75: 507-523.

Jaenisch, R. (1988). Transgenic animals. Science 240: 1468-1474.

Kellum, R. and P. Schedl (1991). A position-effect assay for boundaries of higher order chromosomal domains. Cell 64: 941-950.

Krebs, J. E. and M. Dunaway (1998). The scs and scs′ insulator elements impart a cis requirement on enhancer-promoter interactions. Mol Cell 1: 301-308.

Marshall E. (2002). Clinical research. Gene therapy a suspect in leukemia-like disease. Science. 298:34-35.

Martin, C. C. and R. McGowan (1995). Genotype specific modifiers of transgene methylation and expression in zebrafish, Danio rerio. Genet. Res. Camb. 65: 21028.

Martin, C. C., L. Laforest, M-A. Akimenko and M. Ekker (1999). A role for DNA methylation in gastrulation and somite patterning. Dev. Biol. (in press).

Martienssen, R. (1998). Transposons, DNA methylation and gene control. Trends Genet. 14: 263-264.

McKnight, R. A., A. Shamay, L. Sankaran, R. J. Wall and L. Hennighausen (1992) Matrix-attachment regions can impart position-independent regulation of a tissue-specific gene in transgenic mice. Proc. Natl. Acad. Sci. USA 89: 6943-6947.

Milot, E., F. Fraser and F. Grosveld (1996). Position effects and genetic disease. Trends Genet. 12: 123-126.

Montini, E., P. K. Held, M. Noll, N. Morcinek, M. Al-Dhalimy, M. Finegold, S. Yant, M. Kay and M. Grompe (2002). In vivo correction of murine tyrosinemia type I by DNA-mediated transposition. Mol. Therap. 6: 759-769.

Muir, W. M. and R. D. Howard (2002). Assessment of possible ecological risks and hazards of transgenic fish with implications for other sexually reproducing organisms. Transgenic Res. 11: 101-114.

Niiler, E. (2000). FDA, researchers consider first transgenic fish. Nature Biotech. 18: 143.

O'Neill, R. J. W., M. J. O'Neill and J. A. M. Graves (1998) Undermethylation associated with retroelement activation and chromosome remodelling in an interspecific mammalian hybrid. Nature 393: 68-72.

Pawlowski, W. P. and D. A. Somers (1998). Transgenic DNA integrated into the oat genome is frequently interspersed by host DNA. Proc. Natl. Acad. Sci. USA 95: 12106-12110.

Phi-Van, L., J. P. von Kries, W. Ostertag, and W. H. Stratling (1990). The chicken lysozyme 5′ matrix attachment region increases transcription from a heterologous promoter in heterologous cells and dampens position effects on the expression of transfected genes. Mol. Cell. Biol. 10: 2302-2307.

Pikaart, M. J., F. Recillas-Targa and G. Felsenfeld (1998) Loss of transcriptional activity of a transgene is accompanied by DNA methylation and histone deacetylation and is prevented by insulators. Genes Dev. 12: 2852-2862.

Recillas-Targa, F, AC Bell, G Felsenfeld (1999). Positional enhancer-blocking activity of the chicken β-globin insulator in transiently transfected cells. Proc. Natl. Acad. Sci. USA 96: 14354-14359.

Reichardt, T. (2000). Will souped up salmon sink or swim? Nature 406: 10-12.

Simmen, M. W., S. Leitgeb, J. Charlton, S. J. M. Jones, B. R. Harris, V. H. Clark, and A. Bird (1999). Nonmethylated transposable elements and methylated genes in a chordate genome. Science 283: 1164-1167.

Tweedie, S., K. Charlton, V. Clark, and A. Bird (1997). Methylation of genomes and genes at the invertebrate-vertebrate boundary. Mol. Cell. Biol. 17: 1469-1475.

Yoder, J. A., C. P. Walsh, and T. H. Bestor (1997). Cytosine methylation and the ecology of intragenomic parasites. Trends Genet. 13: 335-340.

Verma, I. (2002). Success and setback: another adverse event. Mol. Ther. 6: 565-566.

Verma, I. M. and N. Somia (1997). Gene therapy—promises, problems and prospects. Nature 389: 239-242.

Winn, R. N., M. B. Norris, K. J. Brayer, C. Torres and S. L. Muller (2000). Detection of mutations in transgenic fish carrying a bacteriophage lambda clI transgene target. Proc. Natl. Acad. Sci. USA 97: 12655-12660.

Yant, S. R., Meuse, W. Chio, Z. Ivics, Z. Izsvak, M. A. Kay (2000) Somatic integration and long-term transgene expression in normal and haemophilic mice using a DNA transposon system. Nature Genetics 25: 35-41.

Yant, SR., A. Ehrhardt, J. G. Mikkelsen, L. Meuse, T. Pham and M. A. Kay (2002). Transposition from a gutless adeno-transposon vector stabilizes transgene expression in vivo. Nature Biotech. 20: 999-1005.

1. A transposon comprising
a transcriptional unit and a plurality of insulator elements, wherein the transcriptional unit is flanked by at least one insulator element on each side of the transcriptional unit, wherein the transcriptional unit comprises an exogenous nucleic acid for introduction into a cell.

2. The transposon of claim 1, wherein the transposon comprises at least two inverted repeat sequences.

3. The transposon of claim 1, wherein the insulator element specifically binds to a CTCF protein.

4. The transposon of claim 1, wherein the insulator element comprises a binding site for a CTCF protein.

5. The transposon of claim 1, wherein the insulator element comprises a 16-base sequence that has at least 80% identity with SEQ ID NO:16.

6. The transposon of claim 1, wherein the insulator element comprises a 16-base sequence that has at least 80% identity with SEQ ID NO:17.

7. The transposon of claim 1, wherein the insulator element comprises a 16-base sequence that has at least 80% identity with SEQ ID NO:18.

8. The transposon of claim 1, wherein the insulator element comprises at least one of SEQ ID NO: 16, SEQ ID NO:17, OR SEQ ID NO:18.

9. The transposon of claim 1, wherein at least one of the insulator elements comprises a scs/scs′ element from the D. melanogangster 87A7 heat shock locus.

10. The transposon of claim 1, wherein at least one of the insulator elements comprises a gypsy moth retrotransposon su(hW) binding region-type insulator.

11. The transposon of claim 1, wherein at least one of the insulator elements comprises an A-element-type insulator.

12. The transposon of claim 1, wherein at least one of the insulator elements comprises an A-element-type insulator from a chicken lysozyme locus.

13. The transposon of claim 1, wherein at least one of the insulator elements comprises an insulator element from a DHS5 site of a chicken β-globulin locus.

14. The transposon of claim 1 wherein at least one of the insulator elements comprises the mammalian apolipoprotein B gene insulator.

15. The transposon of claim 1 wherein at least one of the insulator elements comprises a human β-interferon gene insulator.

16. The transposon of claim 1 wherein at least one of the insulator elements comprises an sns insulator from sea urchin arylsuflatase gene.

17. The transposon of claim 1 wherein at least one of the insulator elements comprises a Drosophila bithorax Fab-7 insulator.

18. The transposon of claim 1 wherein at least one of the insulator elements comprises a mammalian insulator flanking a tyrosinase gene.

19. The transposon of claim 1, wherein the transcriptional unit is disposed between a first insulator element and a second insulator element, and the first insulator element and the second insulator element are disposed between inverted repeats of a transposon.

20. The transposon of claim 1, wherein the transcriptional unit further comprises at least one member of the group consisting of promoters and enhancers.

21. The transposon of claim 1 wherein the exogenous nucleic acid encodes a marker molecule.

22. The transposon of claim 1 wherein the exogenous nucleic acid is a member of the group consisting of DNA encoding an antisense RNA or siRNA.

23. The transposon of claim 1 wherein the exogenous nucleic acid is a member of the group consisting of DNA encoding an mRNA.

24. The transposon of claim 1 comprising at least two inverted repeat sequences that specifically bind to a Sleeping Beauty transposase.

25. The transposon of claim 1 further comprising a suicide sequence nucleic acid.

26. The transposon of claim 13 further comprising an independent promoter for the suicide sequence nucleic acid.

27. A cell, the cell comprising the transposon of claim 1.

28. The cell of claim 27, wherein the cell is in vitro.

29. The cell of claim 27, wherein the cell is in an animal.

30. The cell of claim 27, wherein the cell is in a human.

31. The cell of claim 27, wherein the cell is transfected with the transposon.

32. The cell of claim 27, wherein the cell is electroporated with the transposon.

33. The cell of claim 27, wherein the cell is microinjected with the transposon.

34. The cell of claim 27, wherein the cell is electroporated or microinjected with the transposon, and the cell produces a protein that is encoded by the exogenous nucleic acid.

35. The cell of claim 27, wherein the cell is transfected with the exogenous nucleic acid and produces a protein that is encoded by the exogenous nucleic acid.

36. The cell of claim 27, wherein the cell is a member of the group consisting of lymphocytes, pancreatic cells, neural cells, muscle cells, and blood cells.

37. The cell of claim 27, wherein the cell is a member of the group consisting of hepatocytes, hepatoma cells, primary hepatocytes and liver cells.

38. The cell of claim 27 wherein the cell wherein the cell is a stem cell.

39. The cell of claim 27, wherein the cell wherein the cell is a member of the group consisting of primary pancreatic cells and pancreatic stem cells.

40. The cell of claim 27, wherein the cell wherein the cell is a member of the group consisting of primary hematopoietic cells and hematopoietic stem cells.

41. The cell of claim 32, wherein the protein is a marker.

42. The cell of claim 32, wherein the protein is a therapeutic protein.

43. The cell of claim 34, wherein the therapeutic protein ameliorates a medical condition.

44. An animal, the animal comprising the transposon of claim 1.

45. The animal of claim 44 wherein the animal is a member of the group consisting of a zebrafish, a mouse, and a rat.

46. An animal embryo, the embryo comprising the transposon of claim 1.

47. The animal embryo of claim 46 wherein the embryo is a member of the group consisting of a zebrafish, a mouse, and a rat.

48. A method of altering a cell, the method comprising exposing the cell to a transposon that comprises a transcriptional unit and a plurality of insulator elements, wherein the transcriptional unit is flanked by at least one insulator element on each side of the transcriptional unit, wherein the transcriptional unit comprises an exogenous nucleic acid for introduction into a cell.

49. The method of claim 48 wherein the transposon is introduced into the cell by electroporation.

50. The method of claim 48 wherein the transposon is introduced into the cell by microinjection.

51. The method of claim 48 wherein the cell is a member of the group consisting of lymphocytes, pancreatic cells, neural cells, muscle cells, and blood cells.

52. The method of claim 48 wherein the cell is a member of the group consisting of hepatocytes, hepatoma cells, primary hepatocytes and liver cells.

53. The method of claim 48 wherein the cell is a stem cell.

54. The method of claim 48 wherein the cell is a member of the group consisting of primary pancreatic cells and pancreatic stem cells.

55. The method of claim 48 wherein the cell is a member of the group consisting of primary hematopoietic cells and hematopoietic stem cells.

56. The method of claim 48, wherein the insulator element specifically binds to a CTCF protein.

57. The method of claim 48, wherein the insulator element comprises a binding site for a CTCF protein.

58. The method of claim 48, wherein the insulator element comprises a 16-base sequence that has at least 80% identity with SEQ ID NO: 16.

59. The method of claim 48, wherein the insulator element comprises a 16-base sequence that has at least 80% identity with SEQ ID NO:17.

60. The method of claim 48, wherein the insulator element comprises a 16-base sequence that has at least 80% identity with SEQ ID NO:18.

61. The method of claim 48, wherein the insulator element comprises at least one member of the group consisting of SEQ ID NO:16, SEQ ID NO:17, and SEQ ID NO:18.

62. The method of claim 48, wherein at least one of the insulator elements is chosen from a member of the group consisting of scs/scs′ elements from the D. melanogangster 87A7 heat shock locus, a gypsy moth retrotransposon su(hW)-binding region, an A-element, an A-element from a chicken lysozyme locus, a DHS5 site of a chicken β-globulin locus, a mammalian apolipoprotein B gene insulator, a human β-interferon gene insulator, an sns insulator from sea urchin arylsuflatase gene, a Drosophila bithorax Fab-7 insulator, and a mammalian insulator flanking a tyrosinase gene.

63. The method of claim 48, wherein the transcriptional unit is disposed between a first insulator element and a second insulator element, and the first insulator element and the second insulator element are disposed between at least two inverted repeats.

64. The method of claim 48, wherein the transcriptional unit further comprises at least one member of the group consisting of promoters and enhancers.

65. The method of claim 48 wherein the exogenous nucleic acid encodes a marker molecule.

66. The method of claim 48 wherein the exogenous nucleic acid is a member of the group consisting of antisense DNA, DNA, and cDNA.

67. The method of claim 48 wherein the exogenous nucleic acid encodes siRNA.

68. The method of claim 48 comprising at least two inverted repeat sequences that specifically bind to a Sleeping Beauty transposase.

69. The method of claim 48 further comprising a suicide sequence nucleic acid.

70. The method of claim 69 further comprising an independent promoter for the suicide sequence nucleic acid.

71. The method of claim 48 further comprising exposing a cell to the transposon.

72. The method of claim 71, wherein the cell is in vitro.

73. The method of claim 71, wherein the cell is in an animal.

74. The method of claim 71, wherein the cell is in a human.

75. The method of claim 71, wherein the cell is transfected with the exogenous nucleic acid.

76. The method of claim 71, wherein the cell is electroporated or microinjected with the transposon.

77. The method of claim 71, wherein the cell is transfected with the exogenous nucleic acid and produces a protein that is encoded by the exogenous nucleic acid.

78. The method of claim 77, wherein the protein is a marker.

79. The method of claim 77, wherein the protein is a therapeutic protein.

80. The method of claim 79, wherein the therapeutic protein ameliorates a medical condition.

81. The method of claim 48 further comprising exposing a cell in an animal to the transposon.

82. The method of claim 81 wherein the animal is a member of the group consisting of a zebrafish, a mouse, and a rat.

83. The method of claim 81 wherein the animal is an embryo.

84. The method of claim 83 wherein the embryo is a member of the group consisting of a zebrafish, a mouse, and a rat.

85. A transposon comprising a transcriptional unit and a means for preventing regulation of transcription of host nucleic acid by the transcriptional unit following insertion of into a host mammalian cell nuclear genome.

86. The transposon of claim 85 wherein the host nucleic acid is a gene.

87. The transposon of claim 85 wherein the transcriptional unit comprises an exogenous nucleic acid.

88. The transposon of claim 85, wherein the means for preventing regulation of transcription of host nucleic acid specifically binds to a CTCF protein.

89. The transposon of claim 85, wherein the means for preventing regulation of transcription of host nucleic acid comprises a binding site for a CTCF protein.

90. The transposon of claim 85, wherein the transcriptional unit is disposed between a first insulator element and a second insulator element, and the first insulator element and the second insulator element are disposed between the at least two inverted repeats.

91. The transposon of claim 85, wherein the transcriptional unit further comprises at least one member of the group consisting of promoters and enhancers.

92. The transposon of claim 85 wherein the transcriptional unit encodes a marker molecule.

93. A cell, the cell comprising the transposon of claim 85.

94. The cell of claim 93, wherein the cell is in vitro.

95. The cell of claim 94, wherein the cell is in a human.

96. An animal, the animal comprising the transposon of claim 85.

97. The animal embryo of claim 96 wherein the embryo is a member of the group consisting of a zebrafish, a mouse, and a rat.

98. The transposon of claim 85, wherein the means for preventing regulation of transcription of host nucleic acid comprises a 16-base sequence that has at least 80% identity with SEQ ID NO:16

99. The transposon of claim 85, wherein the means for preventing regulation of transcription of host nucleic acid comprises a 16-base sequence that has at least 80% identity with SEQ ID NO:17

100. The transposon of claim 85, wherein the means for preventing regulation of transcription of host nucleic acid comprises a 16-base sequence that has at least 80% identity with SEQ ID NO:18.

101. The transposon of claim 85, wherein the means for preventing regulation of transcription of host nucleic acid comprises at least one member of the group consisting of SEQ ID NO:16, SEQ ID NO:17, and SEQ ID NO:18.

102. The transposon of claim 85 wherein the transcriptional unit is a member of the group consisting of antisense DNA, DNA, and cDNA.
